SEQUENCE LISTING (21 sequences)

SEQ ID NO: 1 - protein, 7 aa, Artificial
  Peptide that binds to LLC and/or GL261 tumors
HVGGSSV

SEQ ID NO: 2 - protein, 9 aa, Artificial
  Peptide that binds to LLC and/or GL261 tumors
SLRGDGSSV

SEQ ID NO: 3 - protein, 9 aa, Artificial
  Peptide that binds to LLC and/or GL261 tumors
SVRGSGSGV

SEQ ID NO: 4 - protein, 6 aa, Artificial
  Peptide that binds to LLC and/or GL261 tumors
SVGSRV

SEQ ID NO: 5 - protein, 9 aa, Artificial
  Peptide that binds to LLC and/or GL261 tumors
SVVRDGSEV

SEQ ID NO: 6 - protein, 11 aa, Artificial
  Peptide that binds to LLC and/or GL261 tumors
SGRKVGSGSSV

SEQ ID NO: 7 - protein, 9 aa, Artificial
  Peptide that binds to LLC and/or GL261 tumors
SRKQGGTEV

SEQ ID NO: 8 - protein, 4 aa, Artificial
  Peptide that binds to LLC and/or GL261 tumors
SKEK

SEQ ID NO: 9 - protein, 4 aa, Artificial
  conserved subsequence
GSSV

SEQ ID NO: 10 - protein, 7 aa, Artificial
  conserved subsequence
RGDGSSV

SEQ ID NO: 11 - protein, 4 aa, Artificial
  conserved subsequence
GSRV

SEQ ID NO: 12 - protein, 7 aa, Artificial
  conserved subsequence
RGSGSRV

SEQ ID NO: 13 - protein, 7 aa, Artificial
  conserved subsequence
SXRGXGS

SEQ ID NO: 14 - protein, 4 aa, Artificial
  conserved subsequence
GSXV

SEQ ID NO: 15 - protein, 3 aa, Artificial
  conserved sequence
NSV

SEQ ID NO: 16 - protein, 4 aa, Artificial
  conserved subsequence
NSXR

SEQ ID NO: 17 - protein, 4 aa, Artificial
  conserved subsequence
NXVG

SEQ ID NO: 18 - protein, 7 aa, Artificial
  conserved subsequence
RGXGSXV

SEQ ID NO: 19 - protein, 12 aa, Homo sapiens
HHLGGAKQAGDV

SEQ ID NO: 20 - DNA, 20 bp, Artificial
  5′ T7 PCR primer
agcggaccagattatcgcta

SEQ ID NO: 21 - DNA, 19 bp, Artificial
  3′ T7 PCR primer
aaccctcaagacccgttta

(19) United States
(12) Patent Application Publication
(10) Pub. No.: US 2010/0135905 A1
(43) Pub. Date: Jun. 3, 2010

(54) ASSESSMENT OF CANCER SUSCEPTIBILITY TO MOLECULAR TARGETED THERAPY BY USE OF RECOMBINANT PEPTIDES

(76) Inventor: Dennis E. Hallahan, Nashville, TN (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 12/699,450

(22) Filed: Feb. 3, 2010

Related U.S. Application Data
(62) Division of application No. 11/219,634, filed on Sep. 2, 2005.
(60) Provisional application No. 60/606,673, filed on Sep. 2, 2004.

(51) Int. Cl. A61K 51/08; A61K 49/00; A61K 49/04; A61K 49/12; A61K 49/14; A61K 49/16; G01N 33/574; A61P 35/00

(52) U.S. Cl. 424/1.69; 424/9.2; 424/9.1; 424/9.3; 424/9.4; 424/9.6; 435/7.23

(57) ABSTRACT

A method for assessing cancer susceptibility to molecular targeted therapy. Also provided are methods for in vivo panning of diverse molecules for isolation of targeting ligands that specifically bind an apoptotic cell associated with a responding tumor, targeting ligands identified by the panning methods, and diagnostic and imaging uses therefor.

### CROSS REFERENCE TO RELATED APPLICATIONS

This application is based on and claims priority to U.S. Provisional Application Ser. No. 60/606,673, filed Sep. 2, 2004, the disclosure of which is herein incorporated by reference in its entirety.

### GRANT STATEMENT

This work was supported by grants 2R01-CA89674-04 and R01-CA88076-01 from the United States National Institutes of Health. Thus, the U.S. government has certain rights in the presently disclosed subject matter.

### TECHNICAL FIELD

The presently disclosed subject matter generally relates to methods and compositions for assessing cancer susceptibility to molecular targeted therapy. More particularly, the presently disclosed subject matter provides a method for in vivo panning of diverse molecules for isolation of targeting ligands that specifically bind to dead cells associated with a responding tumor. Also provided are novel targeting ligands identified by the panning methods, and diagnostic and imaging uses therefor.

Table of Abbreviations

| | |
|---|---|
| bFGF | basic fibroblast growth factor |
| CPM | counts per minute |
| DiD | 1,1′-dioctadecyl-3,3,3′,3′- |
| | tetramethylindodicarbocyanine perchlorate |
| DiI | 1,1′-dioctadecyl-3,3,3′,3′- |
| | tetramethylindocarbocyanine perchlorate |
| DiO | 3,3′-dilinoleyloxacarboxyanine, perchlorate |
| DTPA | diethylenetriaminepentaacetic acid/acetate |
| DWI | diffusion-weighted imaging |
| EDC | carbodiimide |
| EGFR | epidermal growth factor receptor |
| FGF | fibroblast growth factor |
| FITC | fluorescein isothiocyanate |
| fMRI | functional magnetic resonance imaging |
| Gy | Gray(s) |
| H&E | Hematoxylin & Eosin |
| HCl | hydrochloric acid |
| HMPAO | hexamethylpropylene amine oxime |
| HRP | horseradish peroxidase |
| HUVEC(s) | human umbilical vein endothelial cell(s) |
| i.p. | intraperitoneal |
| IHC | immunohistochemistry |
| LEUR | low energy high-resolution |
| LLC | Lewis lung carcinoma |
| MEM | Modified Eagle Medium |
| MRI | magnetic resonance imaging |
| MRS | proton magnetic resonance spectroscopy |
| MTI | magnetization transfer imaging |
| PBS | phosphate-buffered saline |
| PDGF(R) | platelet derived growth factor (receptor) |
| PET | positron emission tomography |
| PFU | plaque-forming units |
| ROI | region-of-interest |
| RTK(s) | receptor tyrosine kinase(s) |
| SDS | sodium dodecyl sulfate |
| SHNH | succinimidyl 6-hydrazinium nicotinate |
| | hydrochloride |
| SPDP | thiopropionate |
| SPECT | single photon emission computed tomography |
| SQUID | superconducting quantum interference device |
| | magnetometer |
| TBS | Tris-buffered saline |
| TFA | trifluoroacetic acid |
| TKI(s) | RTK inhibitor(s) |
| TMR | tetramethylrhodamine |
| TUNEL | terminal deoxynucleotidyl transferase-mediated |
| | dUTP nick end labeling |
| VEGF(R) | vascular endothelial growth factor (receptor) |
| VLD | vascular length density |
| vWF | von Willebrand Factor |

### BACKGROUND

Specific inhibitors of kinases, including receptor tyrosine kinase (RTK) antagonists, have been used effectively as therapeutic anti-cancer agents, and can enhance the cytotoxic effects of radiation and chemotherapy. RTK inhibitors (TKIs) interrupt signal transduction that is required for cell viability and thereby improve cancer susceptibility to cytotoxic therapy (Geng et al., 2001; Schueneman et al., 2003). TKIs have now entered clinical trials in combination with chemotherapy and radiation therapy for treatment of lung cancer, head and neck cancer, malignant gliomas, and other neoplasms.

Molecular targeted therapy to RTKs that are approved for cancer therapy include HERCEPTIN® (an anti-Her-2/ErbB2 monoclonal antibody), IRESSA® (an epidermal growth factor receptor (EGFR) antagonist), ERBITUXT™ (an anti-EGFR monoclonal antibody), AVASTIN™ (an anti-vascular endothelial growth factor (VEGF) humanized monoclonal antibody), and GLEEVEC® (an antagonist of platelet-derived growth factor receptor (PDGFR) and c-Kit, among others). Unfortunately, each of these produces a response in only a small percentage of patients. Since new TKIs are considered for registration with the FDA every year, rapid assessment of the susceptibility of various cancers to these and other TKIs will minimize the time that a patient will be treated with ineffective or minimally effective cancer therapy before being switched to an alternative regimen.

Additionally, many potential anti-cancer therapeutic molecules, including RTK antagonists and antibodies directed against growth factors, are ineffective or only marginally effective as in vivo therapeutics in subjects. As a result, many cancer patients currently receive therapies that are either completely ineffective or at best only partially effective in treating their conditions. What is needed, then, is a rapid, sensitive assay for determining whether or not a particular therapeutic regimen is effective in a particular patient.

Presently, responses to anti-cancer therapy are measured by assessment of tumor volumes and/or repeated biopsy to analyze pharmacodynamics. These methods of monitoring cancer response are inefficient, however. On the one hand, tumor volume changes often occur independent of therapeutic efficacy when patients are on therapy for prolonged time intervals. Additionally, biopsies are not practical for patients with certain kinds of cancers including, but not limited to brain tumors, lung cancer, pancreatic cancer, and others. And finally, biopsies can result in sampling error so that the response or susceptibility to therapy is not accurately assessed. Thus, improved techniques for monitoring tumor responses to therapy are needed.

To address this need, the presently disclosed subject matter provides methods for identifying ligands that bind to apoptotic cells associated with responding tumors. Such ligands are useful for assessing the susceptibility of tumor cells to molecular targeted therapy, among other applications.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides methods for identifying a molecule that binds a responding tumor in a subject. In some embodiments, the method comprises (a) treating a tumor with at least one of ionizing radiation, a receptor inhibitor, and a receptor tyrosine kinase inhibitor (TKI) to produce a responding tumor; (b) administering to a subject a library of diverse molecules; and (c) isolating one or more molecules of the library from the responding tumor, whereby a molecule that binds a responding tumor is identified. In some embodiments, the methods further comprise subtracting from the library those molecules that bind to the tumor in the absence of exposing the tumor to both ionizing radiation and a tyrosine kinase inhibitor. In some embodiments, the subtracting comprises administering the library to isolated tumor cells or to isolated proteins prior to administering the library to the subject. In some embodiments, the isolated tumor cells are exposed to either ionizing radiation or the tyrosine kinase inhibitor, but not both.

The presently disclosed subject matter also provides methods for identifying a molecule that binds a responding tumor in a subject. In some embodiments, the method comprises (a) exposing a tumor and a control tissue at least one of ionizing radiation, a receptor inhibitor, and a receptor tyrosine kinase inhibitor (TKI) to produce a responding tumor; (b) administering to the tumor and to the control tissue a library of diverse molecules; and (c) detecting one or more molecules of the library that bind to the tumor and that substantially lack binding to the control tissue, whereby a molecule that binds a responding tumor is identified. In some embodiments, the method further comprises (d) isolating the tumor and the control tissue, or fractions thereof; and (e) administering the library to the isolated tumor and to the control tissue, or fractions thereof, in vitro.

The libraries of diverse molecules can be administered to the subject by any mechanism that would result in the members of the libraries coming in contact with the responding tumor. In some embodiments, the administering comprises administering the library by intravascular provision.

Additionally, the administering step is optionally performed at a time at which treatment-inducible antigens are present on the target tissues disclosed herein. In some embodiments, the administering comprises administering the library subsequent to the treating step. In some embodiments, the administering comprises administering the library 0 hours to about 24 hours following the treating step, and in some embodiments the administering comprises administering the library about 4 hours to about 24 hours following the treating step. In some embodiments, the administering comprises administering the library about 24 hours following the treating step.

The isolating step is performed to isolate members of the libraries that have bound to treatment-inducible antigens present on the target tissues disclosed herein. In some embodiments, the isolating is from a biopsy of the tumor. In some embodiments, the isolating step is performed at least about 1 hour subsequent to the treating step. In some embodiments, the isolating step is performed about 24 to about 48 hours subsequent to the treating step.

Any subjects that have tumors that can respond to the treatments disclosed herein by inducing the availability of treatment-inducible antigens on the target tissues disclosed herein can be treated with the compositions and methods disclosed herein. In some embodiments, the subject is a human.

In the practice of the disclosed methods, libraries of diverse molecules are employed for which at least a fraction of the members of the libraries would be expected to bind to the treatment-inducible antigens present on the target tissues disclosed herein. In some embodiments, the library of diverse molecules comprises a library of ten or more diverse molecules. In some embodiments, the library of diverse molecules comprises a library of one hundred or more diverse molecules. And in still other embodiments, the library of diverse molecules comprises a library of a million or more diverse molecules. In some embodiments, the library of diverse molecules comprises a library of molecules selected from the group consisting of peptides, peptide mimetics, proteins, antibodies or fragments thereof, small molecules, nucleic acids, and combinations thereof. In some embodiments, the library of diverse molecules comprises a library of peptides.

In some embodiments, the molecule that binds a responding tumor comprises a ligand that binds a tumor cell, an endothelial cell associated with tumor vasculature, or a blood component. In some embodiments, the molecule binds to a dead cell or to a receptor activated during the physiologic response to the treating step.

In some embodiments of the disclosed methods, each of the exposing, administering, and isolating steps is repeated one or more times.

The presently disclosed subject matter also provides peptides that bind to tumors treated at least one of ionizing radiation, a receptor inhibitor, and a receptor tyrosine kinase inhibitor (TKI) identified by the methods disclosed herein. In some embodiments, the peptide comprises an amino acid sequence as disclosed in one of SEQ ID NOs: 1-18. In some embodiments, the peptide comprises an amino acid sequence of one of SEQ ID NOs: 1-7, 10, and 12. In some embodiments, the peptide comprises an amino acid sequence of SEQ ID NO: 2.

The presently disclosed subject matter also provides methods for detecting a tumor in a subject. In some embodiments, the method comprises (a) treating a suspected tumor with at least one of ionizing radiation, a receptor inhibitor, and a receptor tyrosine kinase inhibitor (TKI); (b) contacting a cell of the suspected tumor with one or more targeting ligands identified by in vivo panning, wherein the one or more targeting ligands comprises a detectable label and binds to a molecule induced on a tumor cell, an endothelial cell associated with tumor vasculature, or a blood component in response to the treating step; and (c) detecting the detectable label, whereby a tumor is detected. In some embodiments, the one or more targeting ligands comprise a peptide comprising an amino acid sequence of any one of SEQ ID NOs: 1-7, 10, and 12, or combinations thereof. In some embodiments, the detectable label is detectable in vivo. In some embodiments, the detectable label comprises a label that can be detected using magnetic resonance imaging, scintigraphic imaging, ultrasound, or fluorescence, such as near infrared emission. In some embodiments, the label that can be detected using scintigraphic imaging comprises a radionuclide label. In some embodiments, the radionuclide label is ¹³¹I or ^{99m}Tc. In some embodiments, the detecting comprises detecting the radionuclide label using positron emission tomography, single photon emission computed tomography, gamma camera imaging, or rectilinear scanning.

The presently disclosed subject matter also provides methods for x-ray-guided selective targeting of a diagnostic composition to a tumor in a subject. In some embodiments, the method comprises (a) treating the tumor with at least one of ionizing radiation, a receptor inhibitor, and a receptor tyrosine kinase inhibitor (TKI); and (b) administering to the subject a diagnostic composition, wherein the diagnostic composition comprises one or more targeting ligands identified by in vivo panning, whereby the diagnostic composition is selectively targeted to the tumor. In some embodiments, the tumor is a primary or a metastasized tumor. In some embodiments, the selective targeting comprises targeting to a responding tumor in the absence of targeting to a non-responding tumor, to non-treated normal tissue, and to irradiated normal tissue. In some embodiments, at least one of the one or more targeting ligands binds to a cell undergoing apoptosis.

The presently disclosed methods can be employed in conjunction with any tumor in a subject. In some embodiments, the tumor is a primary or a metastasized tumor. In some embodiments, the tumor comprises a tumor selected from the group consisting of bladder carcinoma, breast carcinoma, cervical carcinoma, cholangiocarcinoma, colorectal carcinoma, gastric sarcoma, glioma, lung carcinoma, lymphoma, melanoma, multiple myeloma, osteosarcoma, ovarian carcinoma, pancreatic carcinoma, prostate carcinoma, stomach carcinoma, a head, a neck tumor, and a solid tumor. In some embodiments, the tumor is selected from the group consisting of a glioma, a melanoma, and a lung carcinoma.

In some embodiments, the presently disclosed methods further comprise simultaneously detecting two or more tumors in the subject. In some embodiments, the two or more tumors in the subject comprise two or more tumor types. In some embodiments, at least one of the one or more targeting ligands binds to a dead cell or to a molecule induced during a physiologic response to the treating step. In some embodiments, the method further comprises isolating the suspected tumor or a fraction thereof, and the contacting step occurs in vitro.

The presently disclosed subject matter also provides methods for detecting a cell undergoing apoptosis. In some embodiments, the method comprises (a) binding to the cell a reagent that binds to a molecule induced by apoptosis, the reagent comprising: (i) a peptide the binds to a tumor treated with at least one of ionizing radiation, a receptor inhibitor, and a receptor tyrosine kinase inhibitor (TKI), wherein the peptide comprises an amino acid sequence as disclosed in one of SEQ ID NOs: 1-18, and (ii) a detectable marker; and (b) detecting the binding of the reagent to the cell, whereby a cell undergoing apoptosis is detected.

The presently disclosed subject matter also provides methods for assessing the effectiveness of a treatment on a target. In some embodiments, the method comprises (a) contacting the target with a peptide that binds to a tumor treated with at least one of ionizing radiation, a receptor inhibitor, and a receptor tyrosine kinase inhibitor (TKI), wherein the peptide comprises an amino acid sequence as disclosed in one of SEQ ID NOs: 1-18; and (b) determining an extent of binding of the peptide to the target; wherein the extent of binding to the target correlates with the effectiveness of the treatment.

The presently disclosed subject matter also provides methods for noninvasive imaging of a cell undergoing apoptosis. In some embodiments, the methods comprise (a) binding to the cell a reagent that binds to a molecule induced by apoptosis, the reagent comprising: (i) a peptide the binds to a tumor treated with at least one of ionizing radiation, a receptor inhibitor, and a receptor tyrosine kinase inhibitor (TKI), wherein the peptide comprises an amino acid sequence as disclosed in one of SEQ ID NOs: 1-18; and (ii) a contrast agent; and (b) detecting the binding of the reagent to the cell, whereby a cell undergoing apoptosis is imaged.

Treatment of tumors or other targets with ionizing radiation can be accomplished using any dose of radiation that is appropriate. In some embodiments, the treating comprises exposing the tumor to about 2 Gy ionizing radiation or less. In some embodiments, the treating comprises exposing the tumor to at least about 2 Gy ionizing radiation. In some embodiments, the treating comprises exposing the tumor to about 2 Gy to about 6 Gy ionizing radiation. In some embodiments, the treating comprises exposing the tumor to about 2 Gy to about 3 Gy ionizing radiation. In some embodiments, the treating comprises exposing the tumor to about 3 Gy to about 10 Gy ionizing radiation. In some embodiments, the treating comprises exposing the tumor to a dose of ionizing radiation sufficient to increase vascularity within the tumor by at least 5% within 2-48 hours. And in some embodiments, the treating comprises exposing the tumor to ionizing radiation at least about 30 minutes subsequent to providing the tyrosine kinase inhibitor (TKI) to the subject.

Accordingly, it is an object of the presently disclosed subject matter to provide a method for identifying a molecule that binds a responding tumor in a subject. This object is achieved in whole or in part by the presently disclosed subject matter.

An object of the presently disclosed subject matter having been stated above, other objects and advantages will become apparent to those of ordinary skill in the art after a study of the following description of the presently disclosed subject matter and non-limiting Examples.

### BRIEF DESCRIPTION OF THE SEQUENCE LISTING

SEQ ID NOs. 1-8 are amino acid sequences of peptides isolated by the in vivo panning methods disclosed herein that bind to dead cells and/or to receptors activated during the physiologic response to radiation and/or TKI treatment.

SEQ ID NOs: 9-18 are amino acid sequences of conserved motifs identified in the peptides isolated by the in vivo panning methods disclosed herein that bind to dead cells and/or to receptors activated during the physiologic response to therapy.

SEQ ID NO: 19 is an amino acid sequence of a peptide within the human fibrinogen polypeptide that binds to the radiation-induced α_{2B}β₃ receptor.

SEQ ID NOs: 20 and 21 are nucleotide sequences of the primers used to amplify the nucleic acid sequences encoding isolated recombinant phage that bound within irradiated tumors following six rounds of in vivo panning.

### DETAILED DESCRIPTION

### I. Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” mean “one or more” when used in this application, including the claims. Thus, the phrase “an apoptotic cell associated with a responding tumor” refers to one or more apoptotic cells associated with one or more responding tumors.

The term “ligand” as used herein refers to a molecule or other chemical entity having a capacity for binding to a target. A ligand can comprise a peptide, an oligomer, a nucleic acid (e.g., an aptamer), a small molecule (e.g., a chemical compound), an antibody or fragment thereof, a nucleic acid-protein fusion, and/or any other affinity agent. In some embodiments, a ligand is a peptide that binds to an apoptotic cell associated with a responding tumor.

The term “small molecule” as used herein refers to a compound, for example an organic compound, with a molecular weight in one example of less than about 1,000 Daltons, in another example less than about 750 Daltons, in another example less than about 600 Daltons, and in yet another example less than about 500 Daltons. A small molecule also has a computed log octanol-water partition coefficient in the range of about −4 to about +14 in one example, and in the range of about −2 to about +7.5 in another example.

In some embodiments, a small molecule is a peptide mimetic. The term “peptide mimetic” as used herein refers to a ligand that mimics the biological activity of a reference peptide by substantially duplicating the targeting activity of the reference peptide, but it is not a peptide or peptoid. In some embodiments, a peptide mimetic has a molecular weight of less than about 700 Daltons.

The term “target tissue” as used herein refers to an intended site for accumulation of a ligand following administration to a subject. For example, in some embodiments the methods of the presently disclosed subject matter involve a target tissue comprising a responding tumor, and in some embodiments the methods of the presently disclosed subject matter involve a target tissue comprising an apoptotic cell associated with a responding tumor.

As used herein, the phrase “cell associated with a responding tumor” refers to a cell that is altered as a result of exposure to irradiation and/or cytotoxic treatment with a receptor inhibitor or a TKI. In some embodiments, this alteration comprises the cell undergoing apoptosis. Exemplary cells that are associated with a responding tumor include cells of the tumor itself and cells of the tumor's vascular network. This is in contrast to the phrase “tumor-associated cell”, which refers to a cell of a tumor or of the tumor's vascular network under any conditions (i.e. treated or untreated).

The term “control tissue” as used herein refers to a site suspected to substantially lack binding and/or accumulation of an administered ligand. For example, in accordance with the methods of the presently disclosed subject matter, a tumor that has not been treated with both irradiation and a TKI and a non-cancerous tissue are representative control tissues. It should be noted, however, that either ionizing radiation or a TKI alone can under certain conditions result in certain tumor-associated cells undergoing apoptosis. Thus, as used herein, a tumor that has been treated with only one of ionizing radiation or a TKI can be a control tissue despite the possibility that some tumor-associated cells might be undergoing apoptosis.

The terms “target” and “target molecule” as used herein refer to any substance that is specifically bound by a ligand. Thus, the term “target molecule” encompasses macromolecules including, but not limited to proteins, nucleic acids, carbohydrates, lipids, and complexes thereof. In some embodiments, a target is present on or in a responding tumor, and in some embodiments a target is present on or in an apoptotic cell associated with a responding tumor.

The terms “treatment-induced target” and “treatment-induced tumor target” as used herein refer to a target molecule on or in a tumor, the vasculature supplying the tumor, or a blood component, for which at least one of the expression, localization, and ligand-binding capacity of the target molecule are induced by radiation. Such a target molecule can comprise in some embodiments a molecule at the surface of a tumor cell, within a tumor cell, or in the extracellular matrix surrounding a tumor cell. Alternatively, a target molecule can comprise a molecule present at the surface of or within a vascular endothelial cell, or at the surface of or within a blood component such as a platelet or a leukocyte. Treatment-induced targets include, but are not limited to P-selectin, E-selectin, endoglin, α_{2b}β₃ integrin, and αᵥβ₃ integrin.

The term “induce”, as used herein to refer to changes resulting from radiation exposure and/or exposure to a receptor inhibitor or a TKI, encompasses activation of conformational changes in proteins or regulated release of proteins from cellular storage reservoirs to vascular endothelium. Alternatively, induction can refer to a process of conformational change, also called activation, such as that displayed by the glycoprotein IIb/IIIa integrin receptor upon radiation exposure (Staba et al., 2000; Hallahan et al., 2001a). See also U.S. Pat. No. 6,159,443. In some embodiments, the term “induction” refers to the activation of apoptotic cascades that result in the programmed cell death of one or more cells associated with a responding tumor.

The terms “targeting” and “homing”, as used herein to describe the in vivo activity of a ligand (for example, a peptide) following administration to a subject, refer to the preferential movement and/or accumulation of a ligand in a target tissue as compared to a control tissue.

The terms “selective targeting” and “selective homing” as used herein refer to a preferential localization of a ligand (for example, a peptide) that results in an amount of ligand in a target tissue that is in one example about 2-fold greater than an amount of ligand in a control tissue, in another example an amount that is about 5-fold or greater, and in yet another example an amount that is about 10-fold or greater. The terms “selective targeting” and “selective homing” also refer to binding or accumulation of a ligand in a target tissue concomitant with an absence of targeting to a control tissue, in some examples the absence of targeting to all control tissues.

The term “absence of targeting” is used herein to describe no binding or accumulation of a ligand in one or more control tissues under conditions wherein binding or accumulation would be detectable if present. The phrase also is intended to include minimal, background binding or accumulation of a ligand in one or more control tissues under such conditions.

The terms “targeting ligand”, “targeting molecule”, “homing ligand”, and “homing molecule” as used herein refer to a ligand that displays targeting activity. In one example, a targeting ligand displays selective targeting. In some embodiments, a targeting ligand is a peptide that binds to an apoptotic cell.

The term “binding” refers to an affinity between two molecules, for example, a ligand and a target molecule. As used herein, “binding” refers to a preferential binding of one molecule with another in a mixture of molecules. In some embodiments, the binding of a ligand to a target molecule can be considered specific if the binding affinity is about 1×10⁴ M⁻¹ to about 1×10⁶ M⁻¹ or greater.

The phrase “specifically (or selectively) binds”, when referring to the binding capacity of a ligand, refers to a binding reaction which is determinative of the presence of the target in a heterogeneous population of proteins and other biological materials. The phrase “specifically binds” also refers to selectively targeting to responding cells, but not non-responding cells.

The phrases “substantially lack binding” and “substantially no binding”, as used herein to describe binding of a ligand in a control tissue, refer to a level of binding that encompasses non-specific or background binding, but does not include specific binding.

The term “tumor” as used herein refers to both primary and metastasized solid tumors and carcinomas of any tissue in a subject, including but not limited to breast; colon; rectum; lung; oropharynx; hypopharynx; esophagus; stomach; pancreas; liver; gallbladder; bile ducts; small intestine; urinary tract including kidney, bladder and urothelium; female genital tract including cervix, uterus, ovaries (e.g., choriocarcinoma and gestational trophoblastic disease); male genital tract including prostate, seminal vesicles, testes and germ cell tumors; endocrine glands including thyroid, adrenal, and pituitary; skin (e.g., hemangiomas and melanomas), bone or soft tissues; blood vessels (e.g., Kaposi's sarcoma); brain, nerves, eyes, and meninges (e.g., astrocytomas, gliomas, glioblastomas, retinoblastomas, neuromas, neuroblastomas, Schwannomas and meningiomas). The term “tumor” also encompasses solid tumors arising from hematopoietic malignancies such as leukemias, including chloromas, plasmacytomas, plaques and tumors of mycosis fungoides and cutaneous T-cell lymphoma/leukemia, and lymphomas including both Hodgkin's and non-Hodgkin's lymphomas. As used herein, the term “tumor” is intended to refer to multicellular tumors as well as individual neoplastic or pre-neoplastic cells.

As used herein, the phrase “treated tumor” refers to a tumor that has been exposed to at least one of ionizing radiation, a receptor inhibitor, and a receptor tyrosine kinase inhibitor (TKI). As disclosed herein, this treatment can result in the induction of one or more treatment-induced targets on the treated tumor. As disclosed herein, treatment-induced targets are molecules that are induced in response to at least one of ionizing radiation, a receptor inhibitor, and a receptor tyrosine kinase inhibitor (TKI). If the treatment does result in the induction of at least one such treatment-induced target, the treated tumor is also referred to herein as a “responding tumor”.

Accordingly, binding molecules that bind to responding tumors display substantially no binding (e.g., no binding or only background binding) to control tissues. In some embodiments, a tumor that has been exposed to neither ionizing radiation nor a receptor inhibitor or TKI can be a control tissue. In some embodiments, a tumor that does not induce any treatment-induced targets in response to a treatment with at least one of ionizing radiation, a receptor inhibitor, and a receptor tyrosine kinase inhibitor (TKI) can be a control tissue.

The term “subject” as used herein refers to a member of any invertebrate or vertebrate species. The methods of the presently disclosed subject matter are particularly useful for warm-blooded vertebrates. Thus, the presently disclosed subject matter concerns mammals and birds. More particularly contemplated is the detection, diagnosis, and/or imaging of tumors in, as well as the assessment of the effectiveness of anti-tumor treatments in, mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also contemplated is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, contemplated is the detection, diagnosis, and/or imaging of tumors in, as well as the assessment of anti-tumor therapy in, livestock, including but not limited to domesticated swine (pigs and hogs), ruminants, horses, poultry, and the like.

The term “about”, as used herein when referring to a measurable value such as an amount of weight, time, dose (e.g., radiation dose), etc., is meant to encompass variations of in one example ±20% or ±10%, in another example ±5%, in another example ±1%, and in yet another example ±0.1% from the specified amount, as such variations are appropriate to perform the disclosed methods.

The term “isolated”, as used in the context of a nucleic acid or polypeptide (including, for example, a peptide), indicates that the nucleic acid or polypeptide exists apart from its native environment and is not a product of nature. An isolated nucleic acid or polypeptide can exist in a purified form or can exist in a non-native environment.

The terms “nucleic acid molecule” and “nucleic acid” refer to deoxyribonucleotides, ribonucleotides, and polymers thereof, in single-stranded or double-stranded form. Unless specifically limited, the term encompasses nucleic acids containing known analogues of natural nucleotides that have similar properties as the reference natural nucleic acid. The terms “nucleic acid molecule” and “nucleic acid” can also be used in place of “gene”, “cDNA”, and “mRNA”. Nucleic acids can be synthesized, or can be derived from any biological source, including any organism.

### II. General Considerations

RTKs and their ligands have been implicated in angiogenesis, and current data suggest they are potential therapeutic targets. Split-kinase domain RTKs including platelet derived growth factor (PDGF) receptor β, Flk-1/KDR (also known as VEGFR2) and fibroblast growth factor (FGF) receptor play important roles in tumor angiogenesis. The inhibition of vascular endothelial growth factor (VEGF) by antibodies and the use of Flk-1 receptor antagonists have been shown to enhance tumor control when combined with cytotoxic therapy (Prewett et al., 1999; Geng et al., 2001; Gorski at al., 1999). Other RTK ligands, including FGF and PDGF, also appear to contribute to angiogenesis and tumor growth (George, 2001). Basic fibroblast growth factor (bFGF) has been shown to inhibit apoptosis in the microvasculature of mouse lungs and intestines exposed to irradiation (Paris et al., 2001; Fuks at al., 1995). FGF may indirectly contribute to angiogenesis by upregulation of VEGF (Seghezzi at al., 1998). PDGF also increases VEGF secretion in tumor cell lines (Tsai at al., 1995). VEGF, FGF, and PDGF are all up regulated in response to radiation (Gorski et al., 1999; Witte et al., 1989).

The RTK inhibitor (TKI) SU11248 is an orally available indolinone-based synthetic molecule that was identified as a low nM selective inhibitor of the angiogenic receptor tyrosine kinases Flk-1/KDR/VEGFR2 and PDGFRβ in both biochemical and cellular assays (Mendel et al., 2002). SU11248 was also found to inhibit cellular signaling via c-kit and FLT3. SU11248 exhibited broad and potent anti-tumor activity in mice, regressing A431 human epidermoid and Colo205 human colon tumors, arresting the growth of H460 human lung, and substantially delaying the growth of C6 rat and SF763T human glioma xenografts (Mendel et al., 2002).

SU11248 is currently in Phase I clinical trials in patients with advanced cancer. Pharmacokinetic/pharmacodynamic studies in mice have shown that SU11248 inhibited PDGFRβ and Flk-1/KDR/VEGFR2 phosphorylation in a time- and dose-dependent fashion with target plasma concentrations of 50-100 ng/ml. Sustained inhibition of Flk-1/KDRNEGFR2 and PDGFRβ phosphorylation was not required for maximum efficacy, as indicated by the demonstration that target receptor phosphorylation was suppressed for approximately 12 hours at efficacious doses with daily administration (Schueneman at al., 2003). Other recently developed VEGF receptor TKIs in clinical trials include AEE788, PTK787, ZD6474, and SU6668.

Thus, the physiologic responses of receptor inhibitors and/or RTK inhibitors (TKIs) combined with cytotoxic therapy include apoptosis and activation of receptors that participate in physiological responses to blood vessel injury. One model includes VEGF receptor TKIs that enhance the cytotoxic effects of radiation and chemotherapy. This combined therapy results in apoptosis of the tumor endothelium and subsequent activation of inflammation and thrombotic cascades. As disclosed herein, VEGF receptor TKIs enhance the effects of radiation within tumor microvasculature resulting in improved tumor control. Other receptors include, but are not limited to platelet-derived growth factor receptors (PDGFRs), c-kit, fibroblast growth factor receptors (FGFRs), and epidermal growth factor receptors (EGFRs). The nucleic acid and amino acid sequences of several representative, non-limiting examples of these RTKs are available in the GENBANK® database.

Thus, the terms “TKIs” and “receptor inhibitors” encompass inhibitors of signal transduction through these receptors. It is understood, however, that the inhibitors need not necessarily inhibit the functioning of the receptors per se, and also include molecules that inhibit a biological activity of a downstream signaling molecule such that signal transduction via the receptor is inhibited. Representative downstream signaling molecules include, but are not limited to the phosphatidylinositol 3-kinases (PI3Ks), Akt/PKB, and the mammalian target of rapamycin (mTOR). It is also understood that different species of organisms will have different members of these groups of receptors and other signaling molecules, and the instant methods and compositions are not limited to treating just humans. The nucleic acid and amino acid sequences of several representative, non-limiting examples of these signaling molecules are also available in the GENBANK® database.

Cancer susceptibility to TKIs has been evaluated primarily by tumor tissue sectioning and staining. This pharmacodynamic approach is not entirely feasible in patients with brain tumors and primary lung cancer. For that reason, the presently disclosed subject matter relates inter alia to the selection of recombinant peptides from phage-displayed peptide libraries that bind to apoptotic vascular endothelium and/or to epitopes that become accessible in response to anti-tumor therapy. These peptides in turn can be labeled with internal emitters to provide a strategy for non-invasive monitoring of cancer responsiveness to therapy. Typically, the physiologic response to therapy can be seen within 24 hours of therapy, which provides a rapid assessment using non-invasive means.

As disclosed herein, phage displayed peptide libraries can be used to select peptides that bind within responding tumor blood vessels. These peptides can be studied with the intention of monitoring tumor blood vessel response during therapy with receptor inhibitors (e.g., TKIs) and/or radiation. As such, recombinant peptides can bind to cells undergoing apoptosis and provide a strategy to non-invasively monitor cancer response to TKI therapy.

Ionizing radiation induces proteins in tumor vascular endothelium through transcriptional induction and/or posttranslational modification of cell adhesion molecules such as integrins (Hallahan et al., 1995a; Hallahan at at, 1996; Hallahan et al., 1998; Hallahan & Virudachalam, 1999). For example, radiation induces activation of the integrin α_{2b}β₃, also called the fibrinogen receptor, on platelets. The induced molecules can serve as binding sites for targeting ligands.

Although several radiation-induced molecules within tumor blood vessels have been identified and characterized, the α_{2b}β₃ target achieves the greatest peptide binding within responding tumor blood vessels. ¹³¹I-labeled fibrinogen binds specifically to tumors following exposure to ionizing radiation (U.S. Pat. No. 6,159,443). Peptides within fibrinogen that bind to the radiation-induced α_{2b}β₃ receptor include HHLGGAKQAGDV (SEQ ID NO: 19) and the ROD peptide (Hallahan et al., 2001a).

In addition, previous observations of radiation-inducible molecules have employed radiation doses that are sufficient to limit blood flow, as described in Geng et al., 2001; Donnelly at al., 2001; Schueneman at al., 2003; and Lu at aL, 2004. Further, as disclosed therein, a tumor vascular window and Doppler sonography were used to measure the change in tumor blood vessels to determine the response of tumor blood vessels to ionizing radiation. Tumors implanted into the window model developed blood vessels within 1 week. Tumors were then treated with radiation and the response of blood vessels was imaged by use of light microscopy. Radiation doses in the range of 2-3 Gy increased the vascularity within tumors. In contrast, larger doses of radiation such as 6 Gy reduced tumor vascularity. Thus, ligands are sought that demonstrate improved tumor specificity and binding to target molecules induced by reduced radiation doses.

### III. Identification of Ligands that Bind to Responding Tumors and Cells Associated with Responding Tumors

The presently disclosed subject matter provides, inter alia, methods for identifying a molecule (for example a peptide) that binds a responding tumor in a subject. In some embodiments, the method comprise (a) treating a tumor with at least one of ionizing radiation, a receptor inhibitor, and a receptor tyrosine kinase inhibitor (TKI) to produce a responding tumor; (b) administering to a subject a library of diverse molecules; and (c) isolating one or more molecules of the library from the responding tumor, whereby a molecule that binds a responding tumor is identified. In some embodiments of this and other methods disclosed herein, one or more of the exposing, administering, and isolating steps can be repeated one or more times (e.g., 1, 2, 3, 4, 5, 6, 7, 8, 9, or 10 times).

Approaches for optimizing peptide binding affinity and specificity have included the modification of peptide conformation and the addition of flanking amino acids to extend the minimal binding motif. For example, amino acids C-terminal to the RGD sequence are differentially conserved in ROD-containing ligands, and this variation correlates with differences in binding specificity (Cheng et al., 1994; Koivunen et al., 1994). Similarly, cyclization of a prototype RGD peptide to restrict its conformational flexibility improved interaction of the peptide with the vitronectin receptor, yet nearly abolished interaction with the fibronectin receptor (Pierschbacher & Ruoslahti, 1987).

Despite conservation of binding motifs among ligands that bind irradiated tumors and recognition of factors that can influence ligand binding, the identification of peptide sequences for improved targeting activity has thus far relied on high volume screening methods to select effective motifs from peptide libraries (Koivunen et al., 1993; Healy et al., 1995). However, the utility of in vitro-selected peptides is unpredictable in so far as peptide-binding properties are not consistently recapitulated in vivo. To obviate these challenges, the presently disclosed subject matter provides a method for in vivo selection of targeting ligands, described further herein below.

Using the in vivo selection method disclosed herein, novel targeting ligands were identified that can be used for detecting cells undergoing apoptosis or other physiologic responses to therapy. The novel ligands display improved specificity of binding to irradiated tumors and are effective for targeting using low dose irradiation. The disclosed targeting ligands also offer benefits including moderate cost of preparation and ease of handling. Representative peptide ligands are set forth as SEQ ID NOs: 1-7, 10, and 12. Many of the identified peptides also exhibited conserved sequence motifs, which are disclosed as SEQ ID NOs: 5-13 and 17-18. In particular, approximately one-third of identified phage contained the sequence SXRGXGS (SEQ ID NO: 13). Thus, in some embodiments a peptide ligand of the presently disclosed subject matter comprises an amino acid sequence as set forth in any of SEQ ID NOs: 1-18.

### III.A.1. Libraries

As used herein, the term “library” means a collection of molecules. A library can contain a few or a large number of different (referred to herein as “diverse”) molecules, varying from about ten molecules to several billion molecules or more. A molecule can comprise a naturally occurring molecule or a synthetic molecule, which is not found in nature. Optionally, as described further herein below, a plurality of different libraries can be employed simultaneously for in vivo panning.

Representative libraries include, but are not limited to peptide libraries (U.S. Pat. Nos. 6,156,511; 6,107,059; 5,922,545; and 5,223,409), oligomer libraries (U.S. Pat. Nos. 5,650,489 and 5,858,670), aptamer libraries (U.S. Pat. Nos. 6,180,348 and 5,756,291), small molecule libraries (U.S. Pat. Nos. 6,168,912 and 5,738,996), libraries of antibodies and/or antibody fragments (U.S. Pat. Nos. 6,174,708; 6,057,098; 5,922,254; 5,840,479; 5,780,225; 5,702,892; and 5,667,988), libraries of nucleic acid-protein fusions (U.S. Pat. No. 6,214,553), and libraries of any other affinity agent that can potentially bind to a responding tumor (e.g., U.S. Pat. Nos. 5,948,635; 5,747,334; and 5,498,538).

The molecules of a library can be produced in vitro, or they can be synthesized in vivo, for example by expression of a molecule in vivo. Also, the molecules of a library can be displayed on any relevant support, for example, on bacterial pili (Lu et al., 1995) or on phage (Smith, 1985).

A library can comprise a random collection of diverse molecules. Alternatively, a library can comprise a collection of diverse molecules having a bias for a particular sequence, structure, or conformation. See e.g., U.S. Pat. Nos. 5,264,563 and 5,824,483. Methods for preparing libraries containing diverse populations of various types of molecules are known in the art, for example as described in U.S. patents cited hereinabove. Numerous libraries are also commercially available.

In some embodiments of the presently disclosed subject matter, a peptide library can be used to perform the disclosed in vivo panning methods. In one example, a peptide library comprises peptides comprising three or more amino acids, in another example at least five, six, seven, or eight amino acids, in another example ten to twenty amino acids, in another example twenty to fifty amino acids, in another example fifty to 100 amino acids, and in yet another example up to about 200 to 300 amino acids.

The peptides can be linear, branched, or cyclic, and can include non-peptidyl moieties. The peptides can comprise naturally occurring amino acids, synthetic amino acids, genetically encoded amino acids, non-genetically encoded amino acids, and combinations thereof.

A biased peptide library can also be used, a biased library comprising peptides wherein one or more (but not all) residues of the peptides are constant. For example, an internal residue can be constant, so that the peptide sequence is represented as:

(Xaa₁)ₘ−(AA)₁−(Xaa₂)ₙ

wherein Xaa₁ and Xaa₂ are any amino acid, or any amino acid except cysteine, wherein Xaa₁ and Xaa₂ are the same or different amino acids, m and n indicate a number Xaa residues, wherein in some embodiments m and n are independently chosen from the range of 2 residues to 20 residues inclusive, in some embodiments m and n are chosen from the range of 4 residues to 9 residues inclusive, and AA is the same amino acid for all peptides in the library. In one example, AA is located at or near the center of the peptide. More specifically, in one example m and n are not different by more than 2 residues; in another example m and n are equal.

Exemplary so-called sequence biased libraries are those in which AA is tryptophan, proline, or tyrosine. Other exemplary sequence biased libraries are those in which AA is phenylalanine, histidine, arginine, aspartate, leucine, or isoleucine. Still other exemplary sequence biased libraries are those in which AA is asparagine, serine, alanine, or methionine.

A biased library used for in vivo panning can also include a library comprising molecules previously selected by in vitro panning methods. Such in vitro panning methods can be used to selectively remove (i.e. subtract) members of the library that bind to negative control tissues (for example, normal cells or tumors that have not been exposed to treatment with both radiation and a TKI (for example, tumor cells that have been exposed to either ionizing radiation or a TKI), or to isolated proteins) prior to administering the library to the subject. Alternatively, in vitro panning can be used to positively select for members of the library that bind to responding tumors in those instances where a fragment (for example, a biopsy), of the responding tumor can be removed from the subject and contacted with the library in vitro prior to in vivo administration of the positively selected library.

In some embodiments, the library of diverse molecules comprises a library of ten or more molecules. In some embodiments, the library of diverse molecules comprises a library of one hundred or more molecules. In some embodiments, the library of diverse molecules comprises a library of one million or more molecules. In some embodiments, the library of diverse molecules comprises a library of one billion or more molecules.

### III.A.2. Phage Peptide Libraries

In some embodiments of the presently disclosed subject matter, the methods for in vivo panning are performed using a phage peptide library. Phage displayed peptide libraries are a valuable research tool because the amino acid sequence on the capsid is encoded by the recombinant DNA. This DNA can be amplified within bacteria infected with the recombinant bacteriophage. Phage DNA can then be sequenced to determine the amino acid sequence of peptides on the capsid that have been recovered from specific sites such as tumor blood vessels (Ruoslahti, 1996). Phage display is a method to discover peptide ligands while minimizing and optimizing the structure and function of proteins (Smith, 1997; Zwick et al., 1998; Forrer et al., 1999). The phage is used as a scaffold to display recombinant libraries of peptides and provides an approach to recovering and amplifying peptides that bind to putative target molecules in vivo. In vivo selection simultaneously provides positive and subtractive screens because organs and tissues such as tumors are spatially separated. Phage that specifically bind within the vasculature of organs and tissues other than the responding tumor are removed while specific phage homing to responding tumors become enriched through one or more rounds of in vivo and/or in vitro panning.

Phage peptide libraries can be designed so that only linear or only cyclic peptides are displayed. Cyclization can be accomplished in phage-displayed libraries by engineering cysteine residues on both sides of the peptide sequence that is displayed. These cyclic peptide libraries can demonstrate superior affinities for certain targets. For example, when the targets are integrins, one other consideration is the amino acids that follow the RGD sequence such as the serine in fibronectin. Truncations of the fibronectin fragments that bind to integrins cause an alteration in the conformation of the RGD site. This results in altered integrin specificity.

The T7 phage has an icosahedral capsid made of 415 proteins encoded by gene 10 during its lytic phase. The T7 phage display system has the capacity to display peptides up to 15 amino acids in size at a high copy number (415 per phage). Unlike filamentous phage display systems, peptides displayed on the surface of T7 phage are not capable of peptide secretion. T7 phage also replicate more rapidly and are extremely robust when compared to other phage. The stability allows for biopanning selection procedures that require persistent phage infectivity. Accordingly, the use of a T7-based phage display is an aspect of some embodiments of the presently disclosed subject matter. Example 1 describes a representative method for preparation of a T7 phage peptide library that can be used to perform the in vivo panning methods disclosed herein.

A phage peptide library to be used in accordance with the panning methods of the presently disclosed subject matter can also be constructed in a filamentous phage, for example, M13 or an M13-derived phage. In some embodiments, the encoded peptides are displayed at the exterior surface of the phage, for example by fusion to M13 vital protein 8. Methods for preparing M13 libraries can be found in Sambrook & Russell, 2001).

### III.B. In vivo Panning for Ligands that Bind Responding Tumors

The presently disclosed subject matter provides a method for in vivo panning for ligands that bind responding tumors. As used herein, the term “in vivo panning” refers to a method of screening a library for selection of a ligand that homes to an apoptotic cell associated with a responding tumor by administering the library (or a pre-selected fraction thereof) to a subject or to a tissue sample (for example a tumor) isolated from the subject. Thus, the term “in vivo”, as used herein to describe methods of panning or ligand selection, refers to contacting of one or more ligands to endogenous candidate target molecules, wherein the candidate target molecules are naturally present in a subject or a tumor biopsy from a subject, and the contacting occurs in the subject or in the biopsied tumor. By contrast, “in vitro” panning refers to contacting a library of candidate ligands with one or more isolated (for example, via biopsy of a target tissue) or recombinantly produced target molecules.

Thus, in some embodiments a method for in vivo panning as disclosed herein includes the steps of (a) treating a tumor with at least one of ionizing radiation, a receptor inhibitor, and a receptor tyrosine kinase inhibitor (TKI); (b) administering to a subject a library of diverse molecules; (c) procuring the tumor or fraction thereof; and (d) isolating one or more molecules of the library of diverse molecules from the tumor, whereby a molecule that binds a responding tumor is identified. Each step of the method can be sequentially repeated to facilitate ligand selection.

The term “administering to a subject”, when used to describe provision of a library of molecules, is used in its broadest sense to mean that the library is delivered to the responding tumor. For example, a library can be provided to the circulation of the subject by injection or cannulization such that the molecules can pass through the tumor. The mode of administration is not limited to intravascular administration, however, and any other suitable manner of administering the library such that contact between members of the library and tumor-associated cells would be expected to occur can be used with the methods and compositions disclosed herein.

Alternatively or in addition, a library can be administered to an isolated tumor or tumor biopsy. Thus, a method for in vivo panning can also comprise: (a) treating a tumor and a control tissue with at least one of ionizing radiation, a receptor inhibitor, and a receptor tyrosine kinase inhibitor (TKI); (b) administering to the tumor and to the control tissue a library of diverse molecules; (c) detecting one or more molecules of the library that bind to the tumor and that substantially lack binding to the control tissue, whereby a molecule that binds a responding tumor is identified.

The in vivo panning methods of the presently disclosed subject matter can further comprise administering the library to isolated tumor cells or to isolated proteins prior to administering the library to a subject or to a tumor. For example, in vitro panning methods can be performed to select ligands that bind to particular tumor targets, followed by performance of the in vivo panning methods as disclosed herein.

In some embodiments of the presently disclosed subject matter, the radiation treatment comprises administration of about 2 Gy ionizing radiation or less. In other embodiments, the radiation treatment comprises at least about 2 Gy ionizing radiation, optionally about 2 Gy to about 3 Gy ionizing radiation, about 2 Gy to about 6 Gy ionizing radiation, or about 6 Gy to 10 Gy ionizing radiation. In some embodiments, radiation treatment comprises about 10 Gy to about 20 Gy ionizing radiation.

In some embodiments of the presently disclosed subject matter, a library is administered to a tumor-bearing human subject following exposure of the subject to at least one of ionizing radiation, a receptor inhibitor, and a receptor tyrosine kinase inhibitor (TKI). Methods and appropriate doses for administration of a library to a human subject are described in PCT International Publication No. WO 01/09611.

Example 2 describes a representative procedure for in vivo panning of phage-displayed peptide ligands that bind to irradiated tumor vessels in accordance with the presently disclosed subject matter. Briefly, peptide binding was studied in tumor blood vessels of 2 distinct tumor models: (1) GL261 glioma, and (2) Lewis lung carcinoma (LLC). Tumors were irradiated with 3 Gy to facilitate identification of peptide sequences that bind tumors exposed to a minimal dose of ionizing radiation. Phage were administered by tail vein injection into tumor bearing mice following irradiation. Phage were recovered from the tumor thereafter. Following multiple rounds of sequential in vivo binding to irradiated tumors, phage were recovered and individual phage were randomly picked and sequenced. Recovered phage were additionally tested for targeting activity in an animal model of melanoma, as described in Example 4.

### III.C. Recovery of Targeting Ligands

Methods for identifying targeting ligands that bind a responding tumor are selected based on one or more characteristics common to the molecules present in the library. For example, mass spectrometry and/or gas chromatography can be used to resolve molecules that home to a responding tumor. Thus, where a library comprises diverse molecules based generally on the structure of an organic molecule, determining the presence of a parent peak for the particular molecule can identify a ligand that binds to an apoptotic cell associated with a responding tumor.

If desired, a diverse molecule can be linked to a tag, which can facilitate recovery or identification of the molecule. Representative tags are epitope tags (for example, myc tags, FLAG™ tags, His₆ tags, VSV-G tags, HSV tags, V5 tags, or any other tag for which a reagent is available or can be produced to facilitate isolation of the molecule) and small molecules such as biotin. See e.g., Brenner & Lerner, 1992, and U.S. Pat. No. 6,068,829. The presence of these tags allow for the recovery or isolation of the diverse molecules of interest using commercially available reagents (such as anti-epitope tag antibodies, affinity reagents comprising the same, or metal chelators for epitope tags, and avidin- or streptavidin-containing reagents for biotin).

In addition, a tag can be a support or surface to which a molecule can be attached. For example, a support can be a biological tag such as a virus or virus-like particle such as a bacteriophage (“phage”); a bacterium; or a eukaryotic cell such as yeast, an insect cell, or a mammalian cell (e.g., an endothelial progenitor cell or a leukocyte); or can be a physical tag such as a liposome, a microbead, or a nanosphere. A support should optimally have a diameter less than about 10 μm to about 50 μm in its shortest dimension, such that the support can pass relatively unhindered through capillary beds present in the subject and not occlude circulation. In addition, a support can be nontoxic and biodegradable, particularly where the subject used for in vivo panning is not sacrificed for isolation of library molecules from the tumor. Where a molecule is linked to a support, the part of the molecule suspected of being able to interact with a target in a cell in the subject can be positioned so as be able to participate in the interaction.

### III.D. Peptide Ligands

A targeting peptide of the presently disclosed subject matter can be subject to various changes, substitutions, insertions, and deletions where such changes provide for certain advantages in its use. Thus, the term “peptide” encompasses any of a variety of forms of peptide derivatives, that include amides, conjugates with proteins, antibodies cyclized peptides, polymerized peptides, conservatively substituted variants, analogs, fragments, peptoids, chemically modified peptides, and peptide mimetics. The terms “targeting peptide” or “peptide ligand” each refer to a peptide as defined herein above that binds to a responding tumor.

Peptides of the presently disclosed subject matter can comprise naturally occurring amino acids, synthetic amino acids, genetically encoded amino acids, non-genetically encoded amino acids, and combinations thereof. Peptides can include both L-form and D-form amino acids.

Representative non-genetically encoded amino acids include but are not limited to 2-aminoadipic acid; 3-aminoadipic acid; β-aminopropionic acid; 2-aminobutyric acid; 4-aminobutyric acid (piperidinic acid); 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; and ornithine.

Representative derivatized amino acids include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups can be derivatized to form salts, methyl and ethyl esters or other types of esters or hydrazides. Free hydroxyl groups can be derivatized to form O-acyl or O-alkyl derivatives. The imidazole nitrogen of histidine can be derivatized to form N-im-benzylhistidine.

Peptides of the presently disclosed subject matter also include peptides comprising one or more additions and/or deletions or residues relative to the sequence of a peptides for which the sequences are disclosed herein, so long as the requisite targeting activity of the peptide is maintained. The term “fragment” refers to a peptide comprising an amino acid residue sequence shorter than that of a peptide disclosed herein.

Additional residues can also be added at either terminus of a peptide for the purpose of providing a “linker” by which the peptides of the presently disclosed subject matter can be conveniently affixed to a label, solid matrix, or carrier. Amino acid residue linkers are usually at least 1 residue and can be 40 or more residues, more often 1 to 20 residues, but alone do not constitute targeting ligands. Typical amino acid residues used for linking are tyrosine, cysteine, lysine, glutamic and aspartic acid, and the like. In addition, a peptide can be modified by terminal-NH₂ acylation (e.g., acetylation or thioglycolic acid amidation) or by terminal-carboxylamidation (e.g., with ammonia, methylamine, and the like terminal modifications). Terminal modifications are useful, as is well known, to reduce susceptibility by proteinase digestion, and therefore serve to prolong half-life of the peptides in solutions, particularly where the solution is a biological fluid where proteases can be present.

Peptide cyclization is also a useful terminal modification because of the stable structures formed by cyclization and in view of the biological activities observed for such cyclic peptides. An exemplary method for cyclizing peptides is described by Schneider & Eberle, 1993. Typically, tert-butoxycarbonyl protected peptide methyl ester is dissolved in methanol and sodium hydroxide solution is added and the admixture is reacted at 20° C. to hydrolytically remove the methyl ester protecting group. After evaporating the solvent, the tertbutoxycarbonyl-protected peptide is extracted with ethyl acetate from acidified aqueous solvent. The tertbutoxycarbonyl protecting group is then removed under mildly acidic conditions in dioxane cosolvent. The unprotected linear peptide with free amino and carboxyl termini so obtained is converted to its corresponding cyclic peptide by reacting a dilute solution of the linear peptide, in a mixture of dichloromethane and dimethylformamide, with dicyclohexylcarbodiimide in the presence of 1-hydroxybenzotriazole and N-methylmorpholine. The resultant cyclic peptide is then purified by chromatography.

The term “peptoid” as used herein refers to a peptide wherein one or more of the peptide bonds are replaced by pseudopeptide bonds including, but not limited to a carba bond (CH₂—CH₂), a depsi bond (CO—O), a hydroxyethylene bond (CHOH—CH₂), a ketomethylene bond (CO—CH₂), a methylene-oxy bond (CH₂—O), a reduced bond (CH₂—NH), a thiomethylene bond (CH₂—S), a thiopeptide bond (CS—NH), and an N-modified bond (—NRCO—). See e.g., Corringer et al., 1993; Garbay-Jaureguiberry et al., 1992; Tung et al., 1992; Urge et al., 1992; Pavane et al., 1993.

Peptides of the presently disclosed subject matter, including peptoids, can be synthesized by any of the techniques that are known to those skilled in the art of peptide synthesis. Synthetic chemistry techniques, such as a solid-phase Merrifield-type synthesis, can be used for reasons of purity, antigenic specificity, freedom from undesired side products, ease of production, and the like. A summary of representative techniques can be found in Stewart & Young, 1969; Merrifield, 1969; Fields & Noble, 1990; and Bodanszky, 1993. Solid phase synthesis techniques can be found in Andersson et al., 2000, references cited therein, and in U.S. Pat. Nos. 6,015,561; 6,015,881; 6,031,071; and 4,244,946. Peptide synthesis in solution is described by Schröder & Lübke, 1965. Appropriate protective groups usable in such synthesis are described in the above texts and in McOmie, 1973. Peptides that include naturally occurring amino acids can also be produced using recombinant DNA technology. In addition, peptides comprising a specific amino acid sequence can be purchased from commercial sources (e.g., Biopeptide Co., LLC of San Diego, Calif., United States of America, and PeptidoGenics of Livermore, Calif., United States of America).

A peptide mimetic can be designed by: (a) identifying the pharmacophoric groups responsible for the targeting activity of a peptide; (b) determining the spatial arrangements of the pharmacophoric groups in the active conformation of the peptide; and (c) selecting a pharmaceutically acceptable template upon which to mount the pharmacophoric groups in a manner that allows them to retain their spatial arrangement in the active conformation of the peptide. For identification of pharmacophoric groups responsible for targeting activity, mutant variants of the peptide can be prepared and assayed for targeting activity. Alternatively or in addition, the three-dimensional structure of a complex of the peptide and its target molecule can be examined for evidence of interactions, for example the fit of a peptide side chain into a cleft of the target molecule, potential sites for hydrogen bonding, etc. The spatial arrangements of the pharmacophoric groups can be determined by NMR spectroscopy or X-ray diffraction studies. An initial three-dimensional model can be refined by energy minimization and molecular dynamics simulation. A template for modeling can be selected by reference to a template database and will typically allow the mounting of 2-8 pharmacophores. A peptide mimetic is identified wherein addition of the pharmacophoric groups to the template maintains their spatial arrangement as in the peptide.

A peptide mimetic can also be identified by assigning a hashed bitmap structural fingerprint to the peptide based on its chemical structure, and determining the similarity of that fingerprint to that of each compound in a broad chemical database. The fingerprints can be determined using fingerprinting software commercially distributed for that purpose by Daylight Chemical Information Systems, Inc. (Mission Viejo, Calif., United States of America) according to the vendor's instructions. Representative databases include but are not limited to SPREI'95 (InfoChem GmbH of München, Germany), Index Chemicus (ISI of Philadelphia, Pa., United States of America), World Drug Index (Derwent of London, United Kingdom), TSCA93 (United States Environmental Protection Agency), MedChem (Biobyte of Claremont, Calif., United States of America), Maybridge Organic Chemical Catalog (Maybridge of Cornwall, England), Available Chemicals Directory (MDL Information Systems of San Leandro, Calif., United States of America), NCI96 (United States National Cancer Institute), Asinex Catalog of Organic Compounds (Asinex Ltd. of Moscow, Russia), and NP (InterBioScreen Ltd. of Moscow, Russia). A peptide mimetic of a reference peptide is selected as comprising a fingerprint with a similarity (e.g., a Tanamoto coefficient) of at least 0.85 relative to the fingerprint of the reference peptide. Such peptide mimetics can be tested for bonding to a responding tumor using the methods disclosed herein.

Additional techniques for the design and preparation of peptide mimetics can be found in U.S. Pat. Nos. 5,811,392; 5,811,512; 5,578,629; 5,817,879; and 5,817,757; and 5,811,515.

Any peptide or peptide mimetic of the presently disclosed subject matter can be used in the form of a pharmaceutically acceptable salt. Suitable acids which are capable of the peptides with the peptides of the presently disclosed subject matter include inorganic acids such as trifluoroacetic acid (TFA), hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid, and the like.

Suitable bases capable of forming salts with the peptides of the presently disclosed subject matter include inorganic bases such as sodium hydroxide, ammonium hydroxide, potassium hydroxide, and the like, and organic bases such as mono-, di-, and tri-alkyl and aryl amines (e.g., triethylamine, diisopropyl amine, methyl amine, dimethyl amine, and the like), and optionally substituted ethanolamines (e.g., ethanolamine, diethanolamine, and the like).

### IV. Tumor Diagnosis and Imaging

The presently disclosed subject matter further provides methods and compositions for diagnosis and imaging of a tumor in a subject. As used herein, the terms “diagnosis” and “detection”, and grammatical variants thereof, are used interchangeably and refer to the identification of the presence of a tumor in a subject.

Thus, in some embodiments of the presently disclosed subject matter, a composition is prepared, the composition comprising a targeting ligand as disclosed herein and a diagnostic agent. The composition can be used for the detection of a tumor in a subject by: (a) treating a suspected tumor with at least one of ionizing radiation, a receptor inhibitor, and a receptor tyrosine kinase inhibitor (TKI); (b) contacting a cell of the suspected tumor with one or more targeting ligands of the presently disclosed subject matter, wherein the ligand comprises a detectable label; and (c) detecting the detectable label, whereby a tumor is detected. Alternatively, a method for detecting a tumor can comprise: (a) treating a suspected tumor with at least one of ionizing radiation, a receptor inhibitor, and a receptor tyrosine kinase inhibitor (TKI); (b) isolating the suspected tumor, or a fraction thereof; (c) contacting a targeting ligand of the presently disclosed subject matter with the suspected tumor in vitro, wherein the ligand comprises a detectable label; and (d) detecting the detectable label, whereby a tumor is detected.

The presently disclosed subject matter also provides methods for detecting a cell undergoing apoptosis. In some embodiments, the methods comprise (a) binding to the cell a reagent that binds to a molecule induced by apoptosis, the reagent comprising a peptide as disclosed herein and a detectable marker; and (b) detecting the binding of the reagent to the cell, whereby a cell undergoing apoptosis is detected.

The presently disclosed subject matter also provides methods for noninvasive imaging of a cell undergoing apoptosis. In some embodiments, the methods comprise (a) binding to the cell a reagent that binds to a molecule induced by apoptosis, the reagent comprising a peptide as disclosed herein and a contrast agent; and (b) detecting the binding of the reagent to the cell, whereby a cell undergoing apoptosis is imaged.

The presently disclosed subject matter also provides methods for assessing the effectiveness of a treatment on a target. In some embodiments, the methods comprise (a) contacting the target with a peptide as disclosed herein; and (b) determining an extent of binding of the peptide to the target; wherein the extent of binding to the target correlates with the effectiveness of the treatment.

In some embodiments of the presently disclosed method, the binding of the peptide to the target is only detectable when the target is undergoing a physiologic response to therapy including cell death. Thus, in some embodiments, an “extent of binding” refers to an amount of binding that is detectable and is indicative of the target undergoing apoptosis.

In some embodiments of the presently disclosed method, the extent of binding is detectably increased when the target is undergoing apoptosis.

In these embodiments, the extent of binding of the peptide to the target increases as the effectiveness of the treatment increases (i.e. when the treatment causes apoptosis in the target). In these embodiments, where there is some background level of binding of the peptide to the target in the absence of treatment, the extent of binding can be expressed, for example, as a “fold increase over background” after treatment. In some embodiments, a fold increase in labeled peptide binding to a tumor after treatment can be compared to the level of peptide binding to the same tumor prior to treatment.

In order to assess this correlation, an extent of binding can be compared either to an extent determined before initiation of the treatment, or an extent of binding subsequent to a different treatment. In the former case, it can be possible to assess whether the treatment induces apoptosis in the target, and if so, to what degree. In the latter case, it can be possible to compare not only whether a treatment induces apoptosis in the target, but also whether it does so to a greater, lesser, or equivalent extent as the different treatment. In some embodiments of the presently disclosed method, it can be possible to determine whether multiple concurrent or consecutive exposures with the same or different treatments have a synergistic effect relative to single treatments.

Methods for preparation, labeling, delivery, detection/diagnosis, imaging, and treatment effectiveness assessment using targeting ligands of the presently disclosed subject matter are described further hereinbelow.

### IV.A. Conjugation of Targeting Ligands

Antibodies, peptides, or other ligands can be coupled to detectable markers using methods known in the art, including but not limited to carbodiimide conjugation, esterification, sodium periodate oxidation followed by reductive alkylation, and glutaraldehyde crosslinking. See Goldman et al., 1997; Cheng, 1996; Neri et al., 1997; Nabel, 1997; Park et al., 1997; Pasqualini et al., 1997; Bauminger & Wilchek, 1980; U.S. Pat. No. 6,071,890; and European Patent No. 0 439 095.

In addition, a targeting ligand (for example, a peptide) can be recombinantly expressed. For example, a nucleotide sequence encoding a targeting peptide or ligand can be cloned into adenovirus DNA encoding the H1 loop fiber, such that the targeting peptide or ligand is extracellularly presented.

### IV.B. Formulation

In some embodiments, a diagnostic composition, an imaging composition, or a combination thereof, of the presently disclosed subject matter comprises a pharmaceutical composition that includes a pharmaceutically acceptable carrier. Suitable formulations include aqueous and non-aqueous sterile injection solutions that can contain anti-oxidants, buffers, bacteriostats, bactericidal antibiotics, and solutes that render the formulation isotonic with the bodily fluids of the subject; and aqueous and non-aqueous sterile suspensions, which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are sodium dodecyl sulfate (SDS), in some embodiments in the range of 0.1 to 10 mg/ml, in some embodiments about 2.0 mg/ml; and/or mannitol or another sugar, in some embodiments in the range of 10 to 100 mg/ml, in some embodiments about 30 mg/ml; and/or phosphate-buffered saline (PBS). Any other agents conventional in the art having regard to the type of formulation in question can be used.

The methods and compositions of the presently disclosed subject matter can be used with additional adjuvants or biological response modifiers including, but not limited to the cytokines IFN-α, IFN-γ, IL-2, IL-4, IL-6, TNF, or other cytokine affecting immune cells.

### IV.C. Administration

Suitable methods for administration of a diagnostic composition, an imaging composition, or a combination thereof, of the presently disclosed subject matter include, but are not limited to intravascular, subcutaneous, or intratumoral administration. In some embodiments, intravascular administration is employed. For delivery of compositions to pulmonary pathways, compositions can be administered as an aerosol or coarse spray.

For diagnostic applications, a detectable amount of a composition of the presently disclosed subject matter is administered to a subject. A “detectable amount”, as used herein to refer to a diagnostic composition, refers to a dose of such a composition that the presence of the composition can be determined in vivo or in vitro. A detectable amount will vary according to a variety of factors including, but not limited to chemical features of the peptide being labeled, the detectable label, labeling methods, the method of imaging and parameters related thereto, metabolism of the labeled peptide in the subject, the stability of the label (e.g., the half-life of a radionuclide label), the time elapsed following administration of the peptide prior to imaging, the route of administration, the physical condition and prior medical history of the subject, and the size and longevity of the tumor or suspected tumor. Thus, a detectable amount can vary and is optimally tailored to a particular application. After study of the present disclosure, and in particular the Examples, it is within the skill of one in the art to determine such a detectable amount.

In some embodiments, subjects are imaged to detect peptide binding within tumors prior to administration of TKIs. Subjects are then treated with TKIs for 24 to 48 hours. This can be followed by re-administration of labeled peptides. Subjects can then be re-imaged to determine whether there is an increase in labeled peptide binding in tumors following the treatment. This method can be employed to differentiate responding tumors from tumors that are not responding to therapy.

### IV.D. Radiation Treatment

The disclosed targeting ligands are useful for identifying molecules (e.g. peptides) that bind to a responding tumor (e.g. by in vivo or in vitro panning) and for detection and/or imaging of tumors. Panning, detection, and/or imaging of a tumor in a subject can be performed by exposing the tumor to both ionizing radiation and a TKI prior to, concurrent with, or subsequent to administration of a composition of the presently disclosed subject matter (e.g., a library of diverse molecules or a detection/imaging reagent). In accordance with the in vivo panning and detection/imaging methods disclosed herein, the tumor is treated in some embodiments 0 hours to about 24 hours before administration of the library or detection/imaging composition, in some embodiments about 4 hours to about 24 hours before administration of the library or detection/imaging composition, and in some embodiments about 24 hours to about 72 hours before administration of the library or detection/imaging composition. In some embodiments, the tumor is treated about 24 hours before administration of the library or detection/imaging composition.

Low doses of radiation can be used for selective targeting using the peptide ligands disclosed herein. In some embodiments, the dose of radiation comprises about 2 Gy ionizing radiation. Higher radiation doses can also be used, especially in the case of local radiation treatment as described herein below.

Radiation can be localized to a tumor using conformal irradiation, brachytherapy, or stereotactic irradiation. The threshold dose for inductive changes can thereby be exceeded in the target tissue but avoided in surrounding normal tissues. In some embodiments, a dose of about 2 Gy ionizing radiation can be used, in some embodiments a dose of about 2 to about 6 Gy can be used, in some embodiments a dose of about 6 to about 10 Gy can be used, and in some embodiments a dose of about 10 Gy to about 20 Gy ionizing radiation can be used. For treatment of a subject having two or more tumors, local irradiation enables differential dosing at each of the two or more tumors. Alternatively, whole body irradiation can be used, as permitted by the low doses of radiation required for targeting of ligands disclosed herein. Radiotherapy methods suitable for use in the practice of this presently disclosed subject matter can be found in Leibel & Phillips, 1998, among other sources.

### IV.E. Monitoring Distribution In Vivo

In a representative embodiment of the presently disclosed subject matter, a diagnostic and/or imaging composition comprises a label that can be detected in vivo. The term “in vivo”, as used herein to describe imaging or detection methods, refers to generally non-invasive methods such as scintigraphic methods, magnetic resonance imaging, ultrasound, or fluorescence, each described briefly herein below. The term “non-invasive methods” does not exclude methods employing administration of a contrast agent to facilitate in vivo imaging.

The label can be conjugated or otherwise associated with a targeting ligand (e.g., a peptide), a diagnostic agent, an imaging agent, or combinations thereof. Following administration of the labeled composition to a subject, and after a time sufficient for binding, the biodistribution of the composition can be visualized. The term “time sufficient for binding” refers to a temporal duration that permits binding of the labeled agent to an apoptotic cell associated with a responding tumor.

Scintigraphic Imaging. Scintigraphic imaging methods include Single Photon Emission Computed Tomography (SPECT), Positron Emission Tomography (PET), gamma camera imaging, and rectilinear scanning. A gamma camera and a rectilinear scanner each represent instruments that detect radioactivity in a single plane. Most SPECT systems are based on the use of one or more gamma cameras that are rotated about the subject of analysis, and thus integrate radioactivity in more than one dimension. PET systems comprise an array of detectors in a ring that also detect radioactivity in multiple dimensions.

A representative method for SPECT imaging is presented in Example 8. Other imaging instruments suitable for practicing the methods of the presently disclosed subject matter, and instructions for using the same, are readily available from commercial sources. Both PET and SPECT systems are offered by ADAC of Milpitas, Calif., United States of America, and Siemens of Hoffman Estates, Illinois, United States of America. Related devices for scintigraphic imaging can also be used, such as a radio-imaging device that includes a plurality of sensors with collimating structures having a common source focus.

When scintigraphic imaging is employed, the detectable label can comprise a radionuclide label, in some embodiments a radionuclide label selected from the group consisting of ¹⁸F, ⁶⁴Cu, ⁶⁷Cu, ⁶⁷Ga, ⁶⁸Ga, ⁷⁷Br, ^{80m}Br, ⁹⁵Ru, ⁹⁷Ru, ¹⁰³Ru, ¹⁰⁵Ru, ^{99m}Tc, ¹⁰⁷Hg, ²⁰³Hg, ¹²³I, ¹²⁴I, ¹²⁵I, ¹²⁶I, ¹³¹I, ¹³³I, ¹¹¹In, ^{113m}In, ^{99m}Re, ¹⁰⁵Re, ¹⁰¹Re, ¹⁸⁶Re, ¹⁸⁸Re, ^{121m}Te, ^{122m}Re, ^{125m}Te, ¹⁶⁵Tm, ¹⁶⁷Tm, ¹⁶⁸Tm, and nitride or oxide forms derived therefrom. In some embodiments of the presently disclosed subject matter, the radionuclide label comprises ¹³¹I or ^{99m}Tc.

Methods for radionuclide labeling of a molecule so as to be used in accordance with the disclosed methods are known in the art. For example, a targeting molecule (for example, a peptide) can be derivatized so that a radioisotope can be bound directly to it (Yoo et al., 1997). Alternatively, a linker can be added that to enable conjugation. Representative linkers include diethylenetriamine pentaacetate (DTPA)-isothiocyanate, succinimidyl 6-hydrazinium nicotinate hydrochloride (SHNH), and hexamethylpropylene amine oxime (HMPAO; Chattopadhyay at al., 2001; Sagiuchi at al., 2001; Dewanjee at al., 1994; U.S. Pat. No. 6,024,938). Additional methods can be found in U.S. Pat. No. 6,080,384; Hnatowich at al., 1996; and Tavitian at al., 1998.

When the labeling moiety is a radionuclide, stabilizers such as ascorbic acid, gentisic acid, or other appropriate antioxidants can be added to the composition comprising the labeled targeting molecule to prevent or minimize radiolytic damage.

Magnetic Resonance Imaging (MRI). Magnetic resonance image-based techniques create images based on the relative relaxation rates of water protons in unique chemical environments. As used herein, the term “magnetic resonance imaging” refers to magnetic source techniques including conventional magnetic resonance imaging, magnetization transfer imaging (MTI), proton magnetic resonance spectroscopy (MRS), diffusion-weighted imaging (DWI) and functional MR imaging (fMRI). See Rovaris et al., 2001; Pomper & Port, 2000; and references cited therein.

Contrast agents for magnetic source imaging include, but are not limited to paramagnetic or superparamagnetic ions, iron oxide particles (Weissleder et al., 1992; Shen et al., 1993), and water-soluble contrast agents. Paramagnetic and superparamagnetic ions can be selected from the group of metals including iron, copper, manganese, chromium, erbium, europium, dysprosium, holmium, and gadolinium. Exemplary metals are iron, manganese, and gadolinium. In some embodiments, the metal is gadolinium.

Those skilled in the art of diagnostic labeling recognize that metal ions can be bound by chelating moieties, which in turn can be conjugated to a therapeutic agent in accordance with the methods of the presently disclosed subject matter. For example, gadolinium ions are chelated by diethylenetriaminepentaacetic acid (DTPA). Lanthanide ions are chelated by tetraazacyclododocane compounds. See U.S. Pat. Nos. 5,738,837 and 5,707,605. Alternatively, a contrast agent can be carried in a liposome (Schwendener, 1992).

Images derived used a magnetic source can be acquired using, for example, a superconducting quantum interference device magnetometer (SQUID, available with instruction from Quantum Design of San Diego, Calif., United States of America). See U.S. Pat. No. 5,738,837.

Ultrasound. Ultrasound imaging can be used to obtain quantitative and structural information of a target tissue, including a tumor. Administration of a contrast agent, such as gas microbubbles, can enhance visualization of the target tissue during an ultrasound examination. In some embodiments, the contrast agent can be selectively targeted to the target tissue of interest, for example by using a peptide for x-ray guided drug delivery as disclosed herein. Representative agents for providing microbubbles in vivo include but are not limited to gas-filled lipophilic or lipid-based bubbles (e.g., U.S. Pat. Nos. 6,245,318; 6,231,834; 6,221,018; and 5,088,499). In addition, gas or liquid can be entrapped in porous inorganic particles that facilitate microbubble release upon delivery to a subject (U.S. Pat. Nos. 6,254,852 and 5,147,631).

Gases, liquids, and combinations thereof suitable for use with the presently disclosed subject matter include air; nitrogen; oxygen; carbon dioxide; hydrogen; nitrous oxide; an inert gas such as helium, argon, xenon or krypton; a sulphur fluoride such as sulphur hexafluoride, disulphur decafluoride, or trifluoromethylsuiphur pentafluoride; selenium hexafluoride; an optionally halogenated silane such as tetramethylsilane; a low molecular weight hydrocarbon (e.g., containing up to 7 carbon atoms), for example an alkane such as methane, ethane, a propane, a butane, or a pentane, a cycloalkane such as cyclobutane or cyclopentane, an alkene such as propene or a butene, or an alkyne such as acetylene; an ether; a ketone; an ester; a halogenated low molecular weight hydrocarbon (e.g., containing up to 7 carbon atoms); or a mixture of any of the foregoing. Halogenated hydrocarbon gases can show extended longevity, and thus are preferred for some applications. Representative gases of this group include decafluorobutane, octafluorocyclobutane, decafluoroisobutane, octafluoropropane, octafluorocyclopropane, dodecafluoropentane, decafluorocyclopentane, decafluoroisopentane, perfluoropexane, perfluorocyclohexane, perfluoroisohexane, sulfur hexafluoride, and perfluorooctanes, perfluorononanes; perfluorodecanes, optionally brominated.

Attachment of targeting ligands to lipophilic bubbles can be accomplished via chemical crosslinking agents in accordance with standard protein-polymer or protein-lipid attachment methods (e.g., via carbodiimide (EDC) or thiopropionate (SPDP)). To improve targeting efficiency, large gas-filled bubbles can be coupled to a targeting ligand using a flexible spacer arm, such as a branched or linear synthetic polymer (U.S. Pat. No. 6,245,318). A targeting ligand can be attached to the porous inorganic particles by coating, adsorbing, layering, or reacting the outside surface of the particle with the targeting ligand (U.S. Pat. No. 6,254,852). A description of ultrasound equipment and technical methods for acquiring an ultrasound dataset can be found in Coatney, 2001; Lees, 2001; and references cited therein.

Fluorescent Imaging. Non-invasive imaging methods can also comprise detection of a fluorescent label. A targeting ligand comprising a lipophilic component can be labeled with any one of a variety of lipophilic dyes that are suitable for in vivo imaging. See e.g., Fraser, 1996; Ragnarson et al., 1992; and Heredia et al., 1991. Representative labels include, but are not limited to carbocyanine and aminostyryl dyes, for example long chain dialkyl carbocyanines (e.g., 1,1′-dioctadecyl-3,3,3′,3′-tetramethylindocarbocyanine perchlorate (DiO), 3,3′-dilinoleyloxacarboxyanine, perchlorate (DiD), and 1,1¹-dioctadecyl-3,3,3′,3′-tetramethylindodicarbocyanine perchlorate (DID) available from Molecular Probes Inc. of Eugene, Oreg., United States of America) and dialkylaminostyryl dyes. Lipophilic fluorescent labels can be incorporated using methods known to one of skill in the art. For example, VYBRANT™ cell labeling solutions are effective for labeling of cultured cells of other lipophilic components (Molecular Probes Inc. of Eugene, Oreg., United States of America).

A fluorescent label can also comprise sulfonated cyanine dyes, including Cy5.5, Cy5, and Cy7 (available from Amersham Biosciences of Piscataway, N.J., United States of America), IRD41 and IRD700 (available from Li-Cor, Inc. of Lincoln, Nebr., United States of America), NIR-1 (available from Dejindo of Kumamoto, Japan), and La Jolla Blue (available from Diatron of Miami, Fla., United States of America). See also Licha et al., 2000; Weissleder et al., 1999; and Vinogradov et al., 1996.

In addition, a fluorescent label can comprise an organic chelate derived from lanthanide ions, for example fluorescent chelates of terbium and europium (U.S. Pat. No. 5,928,627). Such labels can be conjugated or covalently linked to a targeting ligand as disclosed therein.

For in vivo detection of a fluorescent label, an image is created using emission and absorbance spectra that are appropriate for the particular label used. The image can be visualized, for example, by diffuse optical spectroscopy. Additional methods and imaging systems are described in U.S. Pat. Nos. 5,865,754; 6,083,486; and 6,246,901; among other places.

Near-infrared Emission Spectroscopy. Infrared Emission Spectroscopy can also be employed for imaging using the compositions and methods disclosed herein. In some embodiments, a binding molecule comprises a label that is detectable by near-infrared (NIR) emission spectroscopy.

### IV.F. In Vitro Detection

The presently disclosed subject matter further provides methods for diagnosing a tumor, wherein a tumor sample or biopsy is evaluated in vitro. In this case, a targeting ligand of the presently disclosed subject matter comprises a detectable label such as a fluorescent, epitope, or radioactive label, each described briefly herein below.

Fluorescence. Any detectable fluorescent dye can be used, including but not limited to fluorescein isothiocyanate (FITC), FLUOR X™, ALEXA FLUOR®, OREGON GREEN®, tetramethylrhodamine (TMR), ROX (X-rhodamine), TEXAS RED®, BODIPY® 630/650, and Cy5/5.5/7 (available from Amersham Biosciences of Piscataway, N.J., United States of America, or from Molecular Probes Inc. of Eugene, Oreg., United States of America).

A fluorescent label can be detected directly using emission and absorbance spectra that are appropriate for the particular label used. Common research equipment has been developed for in vitro detection of fluorescence, including instruments available from GSI Lumonics (Watertown, Mass., United States of America), XENOGEN™ Corp. (IVIS® System; Alameda, Calif., United States of America), and Genetic MicroSystems Inc. (Woburn, Mass., United States of America). Most of the commercial systems use some form of scanning technology with photomultiplier tube detection. Criteria for consideration when analyzing fluorescent samples are summarized by Alexay et al., 1996.

Detection of an Epitope. If an epitope label has been used, a protein or compound that binds the epitope can be used to detect the epitope. A representative epitope label is biotin, which can be detected by binding of an avidin-conjugated fluorophore, for example avidin-FITC. Alternatively, the label can be detected by binding of an avidin-horseradish peroxidase (HRP) streptavidin conjugate, followed by colorimetric detection of an HRP enzymatic product. The production of a colorimetric or luminescent product/conjugate is measurable using a spectrophotometer or luminometer, respectively. Other epitope tags that can be employed include, but are not limited to myc tags, FLAG™ tags, His₆ tags, VSV-G tags, HSV tags, and V5 tags.

Autoradiographic Detection. In the case of a radioactive label (e.g., ¹³¹I or ^{99m}Tc) detection can be accomplished by conventional autoradiography or by using a phosphorimager as is known to one of skill in the art. A representative autoradiographic method employs photostimulable luminescence imaging plates (Fuji Medical Systems of Stamford, Conn., United States of America). Briefly, photostimulable luminescence is the quantity of light emitted from irradiated phosphorous plates following stimulation with a laser during scanning. The luminescent response of the plates is linearly proportional to the activity (Amemiya et al., 1988; Hallahan et al., 2001b).

### V. Identification of a Target Molecule

Targeting ligands obtained using the methods disclosed herein can be used to identify and/or isolate a target molecule that is recognized by the targeting ligand. Representative methods include affinity chromatography, biotin trapping, and two-hybrid analysis, each described briefly herein below.

Affinity Chromatography. A representative method for identification of a target molecule is affinity chromatography. For example, a targeting ligand as disclosed herein can be linked to a solid support such as a chromatography matrix. A sample derived from a responding tumor is prepared according to known methods in the art, and such sample is provided to the column to permit binding of a target molecule. The target molecule, which forms a complex with the targeting ligand, is eluted from the column and collected in a substantially isolated form. The substantially isolated target molecule is then characterized using standard methods in the art. See Deutscher, 1990.

Biotin Trapping. A related method employs a biotin-labeled targeting ligand such that a complex comprising the biotin-labeled targeting ligand bound to a target molecule can be purified based on affinity to avidin, which is provided on a support (e.g., beads, a column). A targeting ligand comprising a biotin label can be prepared by any one of several methods, including binding of biotin maleimide (3-(N-maleimidylpropionyl)biocytin) to cysteine residues of a peptide ligand (Tang & Casey, 1999), binding of biotin to a biotin acceptor domain, for example that described in K. pneumoniae oxaloacetate decarboxylase, in the presence of biotin ligase (Julien et al., 2000), attachment of biotin amine to reduced sulfhydryl groups (U.S. Pat. No. 5,168,037), and chemical introduction of a biotin group into a nucleic acid ligand, (Carninci et al., 1996). In some embodiments, a biotin-labeled targeting ligand and the unlabeled same target ligand show substantially similar binding to a target molecule.

Two-Hybrid Analysis. As another example, targeting ligands can be used to identify a target molecule using a two-hybrid assay, for example a yeast two-hybrid or mammalian two-hybrid assay. In some embodiments of the method, a targeting ligand is fused to a DNA binding domain from a transcription factor (this fusion protein is called the “bait”). Representative DNA-binding domains include those derived from GAL4, LEXA, and mutant forms thereof. One or more candidate target molecules are fused to a transactivation domain of a transcription factor (this fusion protein is called the “prey”). Representative transactivation domains include those derived from E. coli B42, GAL4 activation domain II, herpes simplex virus VP16, and mutant forms thereof. The fusion proteins can also include a nuclear localization signal.

The transactivation domain should be complementary to the DNA-binding domain, meaning that it should interact with the DNA-binding domain so as to activate transcription of a reporter gene comprising a binding site for the DNA-binding domain. Representative reporter genes enable genetic selection for prototrophy (e.g., LEU2, HIS3, or LYS2 reporters) or by screening with chromogenic substrates (lacZ reporter).

The fusion proteins can be expressed from a same vector or different vectors. The reporter gene can be expressed from a same vector as either fusion protein (or both proteins), or from a different vector. The bait, prey, and reporter genes are co-transfected into an assay cell, for example a microbial cell (e.g., a bacterial or yeast cell), an invertebrate cell (e.g., an insect cell), or a vertebrate cell (e.g., a mammalian cell, including a human cell). Cells that display activity of the encoded reporter are indicative of a binding interaction between the peptide and the candidate target molecule. The protein encoded by such a clone is identified using standard protocols known to one of skill in the art.

Additional methods for yeast two-hybrid analysis can be found in Brent & Finley, 1997; Allen et al., 1995; Lecrenier et al., 1998; Yang et al., 1995; Bendixen at al., 1994; Fuller et al., 1998; Cohen at al., 1998; Kolonin & Finley, 1998; Vasavada et al., 1991; Rehrauer et al., 1996; and Fields & Song, 1989.

### EXAMPLES

The following Examples have been included to illustrate modes of the presently disclosed subject matter. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Preparation of a Phaqe Recombinant Peptide Library

A population of DNA fragments encoding recombinant peptide sequences was cloned into the T7 SELECT® vector (Novagen Brand, a unit of EMD Biosciences, Inc., Madison, Wis., United States of America). Cloning at the Eco RI restriction enzyme recognition site places the recombinant peptide in-frame with the 10B protein such that the peptide is displayed on the capsid protein. The resulting reading frame requires an AAT initial codon followed by a TCX codon.

The molar ratio between insert and vector was 1:1. Size-fractionated cDNA inserts were prepared by gel filtration on SEPHAROSE™ 4B and ranged from 27 base pairs to 33 base pairs. cDNAs were ligated by use of the DNA ligation kit (Novagen Brand, a unit of EMD Biosciences, Inc., Madison, Wis., United States of America). Recombinant T7 DNA was packaged according to the manufacturer's instructions and amplified prior to biopanning in animal tumor models. The diversity of the library was 10⁷.

### Example 2

### In vivo Panning for Peptide Ligands

GL261 murine glioma cells and Lewis lung carcinoma (LLC) cells were implanted into the hind limb of C57BL/6 mice (see Hallahan et al., 1995b; Hallahan et al., 1998; Hallahan & Virudachalam, 1999).

To determine the optimal time at which peptides bind within tumors, phage were administered at 1 hour before, at 1 hour after, and at 4 hours after irradiation of both LLC and GL261 tumors. Phage were recovered from tumors when administered 4 hours after irradiation. Phage administered 1 hour before or 1 hour after irradiation were not recovered from tumors. These data indicate that the optimal time of administration is beyond 1 hour after irradiation.

For in vivo panning, tumors were irradiated with 3 Gy and approximately 10¹⁰ phage (prepared as described in Example 1) were administered by tail vein injection into each of the tumor bearing mice at 4 hours following irradiation. Tumors were recovered at one hour following injection and amplified in BL21 bacteria. Amplified phage were pooled and re-administered to a tumor-bearing mouse following tumor irradiation. The phage pool was sequentially administered to a total of 6 animals. As a control, wild type phage lacking synthetic peptide inserts were identically administered to a second experimental group of animals.

To determine the titer of phage binding in a tumor or in normal tissue, recovered phage were amplified in BL21 bacteria. Bacteria were plated and the number of plaques present was counted. To determine the total phage output per organ, the number of plaque forming units (PFU) on each plate was divided by the volume of phage plated and the weight of each organ. Normal variation was observed as a 2-fold difference in PFU. In the present Example, background binding within tumor blood vessels was approximately 10⁴ phage. Phage that bound to the vasculature within irradiated tumors show enrichment in the tumor relative to other organs and enrichment in the irradiated tumor relative to the control phage without DNA insert. Phage that home to irradiated tumors showed a background level of binding in control organs that was lower than control phage without DNA insert.

Following six rounds of in vivo panning, fifty recombinant phage peptides that bound within irradiated tumors were randomly selected for further analysis. The nucleic acid sequence encoding recombinant phage was amplified by PCR using primers set forth as SEQ ID NOs: 20-21 (available from Novagen Brand, a unit of EMD Biosciences, Inc., Madison, Wis.). An individual phage suspension was used as template. Amplified peptides were sequenced using an ABI PRISM® 377 sequencer (Applied Biosystems of Foster City, Calif., United States of America). The sequences of the encoded peptides are listed in Table 1. Several conserved subsequences were deduced from the recovered peptides and are presented in Table 2.

TABLE 1
Peptides Identified by In vivo
Panning of LLC and GL261 Tumors

| | | |
|---|---|---|
| | Phage Recovered | Phage Recovered |
| Peptide | from LLC tumors | from GL261 tumors |
| Sequence^{a} | (Frequency) | (Frequency) |

Experiment A

| | | |
|---|---|---|
| HVGGSSV | 7 | 12 |
| (SEQ ID NO: 1) | (28%) | (48%) |
| SLRGDGSSV | 7 | 2 |
| (SEQ ID NO: 2) | (28%) | (8%) |
| SVRGSGSGV | 7 | 0 |
| (SEQ ID NO: 3) | (28%) | (0%) |
| SVGSRV | 1 | 3 |
| (SEQ ID NO: 4) | (4%) | (12%) |
| Unique Sequences | 3 | 8 |
| | (12%) | (32%) |

Experiment B

| | | |
|---|---|---|
| SVVRDGSEV | 3 | (not determined) |
| (SEQ ID NO: 5) | (21%) | |
| SLRGDGSSV | 2 | (not determined) |
| (SEQ ID NO: 2) | (14%) | |
| SGRKVGSGSSV | 7 | (not determined) |
| (SEQ ID NO: 6) | (50%) | |
| SRKQGGTEV | 1 | (not determined) |
| (SEQ ID NO: 7) | (7%) | |
| SKEK | 1 | (not determined) |
| (SEQ ID NO: 8) | (7%) | |

Note:
^{a}all peptides identified include an N-terminal asparagine (N) residue encoded by the vector.

TABLE 2
Conserved Motifs within Peptides
Identified by In vivo Panning

| | | Frequency of |
|---|---|---|
| Conserved Sequence | | Recovery |
| GSSV | (SEQ ID NO: 9) | 58% |
| SXRGXGS | (SEQ ID NO: 13) | 28% |
| GSXV | (SEQ ID NO: 14) | 80% |
| N-terminal NSV | (SEQ ID NO: 15)^{a} | 22% |
| N-terminal NSXR | (SEQ ID NO: 16)^{a} | 39% |
| N-terminal NXVG | (SEQ ID NO: 17)^{a} | 34% |

Note:
^{a}all peptides identified include an N-terminal asparagine (N) residue encoded by the vector.

Peptide sequences recovered from both tumor types include HVGGSSV (SEQ ID NO: 1), SLRGDGSSV (SEQ ID NO: 2), and SVGSRV (SEQ ID NO: 4). Of the peptide sequences recovered from several irradiated tumors, 58% had the subsequence GSSV (SEQ ID NO: 9), 28% had the sequence RGDGSSV (SEQ ID NO: 10), and 6% had the sequence GSRV (SEQ ID NO: 11). Approximately 22-40 of 10⁶ injected phage were recovered from irradiated tumors having a peptide insert comprising the subsequence GSSV (SEQ ID NO: 9). By contrast, no phage were from irradiated tumors following administration of 10⁶ wild type phage. In a separate experiment, additional peptide sequences isolated from responding tumors include SWRDGSEV (SEQ ID NO: 5), SGRKVGSGSSV (SEQ ID NO: 6), SRKQGGTEV (SEQ ID NO: 7), and SKEK (SEQ ID NO: 8).

The amino acid sequences of all phage that were recovered from both tumors were studied in order to identify homologous sequences (Table 2). The most commonly recovered phage peptide had amino acid sequence HVGGSSV (SEQ ID NO: 1), and the second most common sequence was SLRGDGSSV (SEQ ID NO: 2). The probability of recovering these peptide sequences from both tumor subtypes is 625/10¹⁴ for each of the peptide sequences. The peptide sequence GSSV (SEQ ID NO: 9) was present in 58% of the phage recovered from tumors. Homology between peptides recovered from LLC and GL261 included 100% homology in SLRGDGSSV (SEQ ID NO: 2) and 70% homology in RGSGSRV (SEQ ID NO.: 12). Of interest is a 6 amino acid homology spanning over 8 amino acids that include amino acids SXRGXGS (SEQ ID NO: 13), which was recovered from 28% of all phage in 2 tumor models (p<0.0001). The probability of having six identical amino acids by chance is 6⁻²⁴.

### Example 3

### Binding of SEQ ID NO: 6 to Treated LLC Tumor Blood Vessels

The amino acid sequence RGXGSXV (SEQ ID NO: 18) was found in 41% of phage recovered from treated LLC tumors. To determine the pattern of RGDGSSV (SEQ ID NO: 10) peptide binding within responding tumor blood vessels, biotinylated peptide was administered by tail vein injection. Tumors were implanted into both hind limbs of mice. The right tumor was irradiated according to Example 2, and the left served as an untreated internal negative control. Biotinylated peptide was administered by tail vein injection immediately prior to tumor irradiation. Fluorescent microscopy of FITC-conjugated avidin staining of biotinylated peptide showed accumulation throughout the lumen of responding tumors as compared to the near absence of binding in untreated control tumors.

### Example 4

### Peptide Targeting in Additional Tumors

The binding properties of phage encoding HVGGSSV (SEQ ID NO: 1), SLRGDGSSV (SEQ ID NO: 2), SVRGSGSGV (SEQ ID NO: 3), and SVGSRV (SEQ ID NO: 4) were additionally characterized in a B16F0 melanoma model. Peptides set forth as SEQ ID NOs: 1 and 2 bound within the melanoma, lung carcinoma, and glioma tumor models. SEQ ID NO: 3 bound within glioma and melanoma, and SEQ ID NO: 4 bound within lung carcinoma and glioma.

### Example 5

### Characterization of Peptide Binding to Irradiated Tumors

To determine where recombinant peptides bind in tumor blood vessels, the biodistribution of biotinylated peptides was assessed. Tumors were treated with 3 Gy and biotinylated peptides were administered by tail vein at 4 hours following irradiation. Tumors were recovered 30 minutes following administration of biotinylated peptides. Tumors were snap frozen and sectioned on a cryostat. Frozen sections were then incubated with an avidin- fluorescein isothiocyanate (FITC) conjugate and imaged by fluorescent microscopy. Recombinant peptides (for example, those set forth in Table 1) were observed to bind the vascular endothelium within tumor blood vessels.

An anti-α_{2b}β₃ monoclonal antibody was administered by tail vein to determine whether this receptor is required for recombinant phage binding in irradiated tumors. Phage encoding SLRGDGSSV (SEQ ID NO: 2) on the capsid protein were injected immediately after blocking antibody or control antibody. Phage were recovered from the tumor and controls organs and quantified by plaque formation. Radiation induced a 4-fold increase in phage binding in tumor. Blocking antibody eliminated induction of phage binding, while control antibody to P-selectin (on activated platelets) did not reduce phage binding. Thus, the tumor binding activity of targeting peptide SLRGDGSSV (SEQ ID NO: 2) is dependent on its interaction with the α_{2b}β₃ receptor.

### Example 6

### Development of Peptides to Inducible Receptors

Phage-displayed peptides recovered from responding tumors include the amino acid sequence arginine-glycine-aspartic acid (RGD). Proteins that bind the RGD peptide include the β₁, β₃, and β₅ chains of integrins, which heterodimerize with the αv chain to form the αᵥβ₃ integrin on the endothelium or with the α_{2b} chain on platelets (Ruoslahti, 1996). To determine whether the level of these integrins increases in response to therapy, immunohistochemical staining was used to study integrins in responding tumors.

GL261 murine gliomas were implanted into the hind limb of C57BL/6 mice. Tumors were grown to a diameter of 10-12 mm over 8-10 days, followed by irradiation (6 Gy). Six hours after irradiation, tumors were dissected and fixed. Immunohistochemical staining for integrin α_{2b}β₃ and the αv chain of integrin αᵥβ₃ revealed increased levels of the β₃ chain and the α_{2b} chain within the lumen of the microvasculature of tumors isolated 6 hours after therapy, but no increase in untreated control tumors.

### Example 7

### Kinetics of Integrin Induction in Irradiated Endothelial Cells

Flow cytometry analysis of β₃ integrin expression in HUVECs after irradiation was performed. HUVECs were irradiated with 3 Gy and fluorescent-labeled β₃ antibody was added to cells at 0, 1, 6, 24, and 48 hours. Increased antibody binding at 6, 24, and 48 hours following therapy was observed, whereas the one hour time point showed no increased binding.

### Example 8

### Clinical Trials of X-Ray-Guided Delivery Using a Peptide Ligand

Ligand Preparation and Administration. Bibapcitide (ACUTECT™, available from Diatide, Inc. of Londonderry, N.H., United States of America) is a synthetic peptide that binds to GP-IIb/IIIa receptors on activated platelets (Hawiger et al., 1989; Hawiger & Timmons, 1992). Bibapcitide was labeled with ^{99m}Tc in accordance with a protocol provided by Diatide Inc.

Reconstituted ^{99m}Tc-labeled bibapcitide was administered to patients at a dose of 100 μg of bibapcitide radiolabeled with 10 mCi of ^{99m}Tc. Patients received ^{99m}Tc-labeled bibapcitide intravenously immediately prior to irradiation. Patients were then treated with 10 Gy or more. Patients underwent gamma camera imaging prior to irradiation and 24 hours following irradiation.

Following planar image acquisition, those patients showing uptake in irradiated tumors underwent tomographic imaging using SPECT and repeat imaging at 24 hours. Patients showing no uptake on planer images during this 24-hour time frame had no further imaging. Each patient had an internal control, which consisted of a baseline scan immediately following administration of ^{99m}Tc-labeled bibapcitide.

Patients were treated with X-irradiation ranging from 4 to 18 MV photon using external beam linear accelerator at Vanderbilt University. Appropriate blocks, wedges, and bolus to deliver adequate dose to the planned target volume was utilized. The site of irradiation, treatment intent, and normal tissue considerations determined the radiation dosage and volume. When stereotactic radiosurgery was used, the dose was prescribed to the tumor periphery.

Image Analysis. Image acquisition consisted of both planar and single photon emission computed tomography (SPECT) studies. Planar studies were performed on a dual-head gamma camera (Millennium VG-Variable Geometry model available from General Electric Medical Systems of Milwaukee, Wis., United States of America) equipped with low energy high-resolution (LEUR) collimators. This type of collimator represents a compromise between sensitivity (photon counting efficiency) and image resolution. Planar nuclear medicine images were acquired with a 256×256 acquisition matrix (pixel size approximately 0.178 cm/pixel) for 10 minutes. In order to maximize collimator-gamma camera system sensitivity the source-to-detector surface distance was minimized to the extent that patient geometry allows. The spatial distribution of fibrinogen within the planar image was measured using region-of-interest (ROI) analysis. Two different size ROIs (5×5 pixel, and 15×15 pixel) was used in both the tumor and surrounding organs and tissues in the patient. The rationale for using ROIs with different dimensions is to be able to quantify image counts while at the same time isolating any possible influence of ROI size on the results. Tumor-to-background ratios were computed as the ratio of average counts in the tumor region divided by average counts in surrounding organs and tissues, each corrected for background. Background counts was determined based on ROI analysis of a separate planar acquisition performed in the absence of a radioactive source.

Three-dimensional nuclear medicine SPECT examinations were performed using the same dual-head gamma camera system. Each SPECT study comprised a 360 scan acquired with a step-and-shoot approach utilizing the following acquisition parameters: three increments between views, a 256×256×64 acquisition matrix, LEUR collimation and 60 seconds per view. Images were reconstructed using analytical filtered back-projection and statistical maximum likelihood techniques with photon attenuation correction and post-reconstruction deconvolution filtering for approximate detector response compensation. In this case, correction for background consisted of subtracting counts acquired in a single 60-second planar view from all views of the SPECT projection data prior to image reconstruction. SPECT tumor-to-background ratios were computed using quantitative ROI techniques identical to the planar studies.

Dose De-escalation Study. To determine whether the ^{99m}Tc-RGD peptidomimetic binds within all responding tumors, targeting was studied in patients with gliomas, breast carcinoma, lung carcinoma, meningiomas, and pituitary adenomas. A dose de-escalation study was conducted in which the radiation dose was reduced to 5 Gy, which was not sufficient for RGD-peptidomimetic binding to responding tumors.

Results. Administration of ^{99m}Tc-labeled bibapcitide, an RGD peptide mimetic, immediately prior to radiation resulted in tumor binding in 4 of 4 patients (Hallahan et al., 2001a). Two patients among this group had second neoplasms that were not treated with radiation, and binding of ^{99m}Tc-labeled bibapcitide was not observed in the non-responding tumors. Administration of the ^{99m}Tc-labeled bibapcitide within one hour following radiation also failed to show localization of the targeting molecule to the tumor (Hallahan et al., 2001a).

### Discussion of Example 8

The clinical study disclosed in Example 8 demonstrated three general findings. First, it is feasible to monitor cancer response by use of peptides that bind to inducible receptors. Second, the dose of radiation required to activate the receptor is 10 Gy when tumors are treated without VEGF receptor TKIs. As disclosed herein, VEGFR TKIs reduce the threshold of peptide binding to 2 Gy. And third, the RGD peptidomimetic achieves non-specific binding, which emphasizes the importance of the improving the specificity of binding by recombinant peptides.

### Example 9

### VEGF Receptor TKIs Enhance Radiation-Induced Apoptosis in Endothelium

To determine whether broad spectrum RTK inhibition enhances the cytotoxic effects of radiation on vascular endothelium, HUVECs were treated with either 100 nM SU11248 or vehicle, incubated for 30 minutes, and treated with radiation (6 Gy). After a 24-hour incubation period, cells were fixed and stained with Hematoxylin and Eosin (H&E). Five high-powered fields (400×) were observed and counted for each experimental group. The percentage of endothelial cells demonstrating apoptotic nuclei 24 hours post treatment was determined for each experimental group. Untreated control cells show 2% apoptotic nuclei as compared to 7% and 8% after treatment with SU11248 or radiation, respectively (p>0.1). HUVECs treated with SU11248 followed by 6 Gy showed 21% of cells with apoptotic nuclei at 24 hours, which was significantly greater than either agent alone (p<0.02) or untreated control cells (p<0.001).

### Example 10

### Clonogenic Survival of HUVECs

To determine whether enhanced apoptotic response in endothelial cells treated with SU11248 results in reduced clonogenic cell survival, HUVECs were subcultured and colony formation was quantified. Tumor vasculature was observed before and 48 hours after treatment with SU11248, 3 Gy, and SU11248+3 Gy. Five mice were treated in each of the treatment groups. HUVECs treated with SU11248 prior to irradiation showed a significant reduction in clonogenic survival as compared to radiation alone (p<0.05). This induction of apoptosis correlated with the biological response in tumor blood vessels and tumor growth delay (Schueneman et al., 2003; Lu et al., 2004). Growth factors produced by tumors could enhance the viability of tumor vascular endothelium.

### Example 11

### TKI-enhanced Radiation-induced Destruction of Tumor Vasculature

To determine whether SU11248 enhances radiation-induced destruction of tumor vasculature, SU11248 (40 mg/kg) was administered to mice prior to irradiation with 3 Gy. Tumor vascular linear density was measured by use of intravital tumor vascular window. Observations of tumor vasculature before and 48 hours after treatment with SU11248, 3 Gy, or SU11248 followed by 3 Gy indicated that RTK inhibition increased tumor vascular destruction as compared to either agent alone. Five mice were treated in each of the treatment groups, and the vascular length density after treatment was quantified. Within 72 hours, vascular length density (VLD) in tumors was significantly reduced to 8% of that at 0 hours (p<0.01). In comparison, tumors treated with either 3 Gy or SU11248 alone showed an insignificant reduction in vascular length density to 75 and 84% that of 0 hour, respectively. Combined treatment with SU11248 and 3 Gy achieved significant reduction in VLD as compared to either agent alone.

### Example 12

### Pharmacodynamics of VEGF Receptor TKIs

To study the pharmacodynamics of SU11248 combined with cytotoxic therapy, tissue sections from tumors treated with radiation, SU11248, or SU11248 followed by irradiation was analyzed by terminal deoxynucleotidyl transferase-mediated dUTP nick end labeling (TUNEL) staining. Tumors treated with SU11248 alone or radiation alone developed no TUNEL staining, whereas SU11248 followed by 3 Gy resulted in positive TUNEL staining in endothelial cells (determined by co-localization with von Willebrand Factor; vWF).

### Example 13

### Enhancement of Tumor Growth Delay by TKI Exposure

As disclosed herein, the induction of apoptosis correlated with the biological response in tumor blood vessels and tumor growth delay. To determine whether SU11248 enhances tumor growth delay in irradiated tumors, mice bearing LLC and GL261 hind limb tumors were treated daily with i.p. injection of 40 mg/kg SU11248 or drug vehicle 30 minutes before each 3 Gy dose of radiation (total of seven administrations of each of SU11248 and radiation). Both the inhibitor and radiation were discontinued after day 8. The mean fold increases in tumor volumes in five mice in each of the treatment groups (vehicle, SU11248, 21 Gy, and SU11248+21 Gy) were determined. Time to doubling of LLC tumor size was 5, 6, 8, and 16 days for each group, respectively. Tumors showed a significant increase in tumor growth delay when SU11248 was added before daily 3 Gy fractions as compared with either agent alone (p=0.05).

### Example 14

### Responses to Other VEGF Receptor TKIs

Other VEGF receptor inhibitors, SU5416 and SU6668, also induced apoptosis within tumor vascular endothelium (Geng et al., 2001; Lu et al., 2004). Tumor vascular windows of LLC tumors at 96 hours following treatment with 3 Gy alone, SU6668 alone, or SU6668 and 3 Gy were examined. The tumor vasculature responded with apoptosis of endothelial cells and destruction of blood vessels (Lu et al., 2004).

This induction of apoptosis also correlated with the biological response in tumor blood vessels and tumor growth delay. Tumor vascular response to combined TKI and cytotoxic therapy correlated with tumor control (Geng et al., 2001; Schueneman at al., 2003; Lu et al., 2004).

### Discussion of Examples 9-14

The response of tumor blood vessels to RTK inhibitors can be studied by use of MRI and Doppler ultrasound. See e.g., Donnelly et al., 2001; Geng et al., 2001; Schueneman et al., 2003). Dynamic contrast enhanced MRI has been used to evaluate the response to VEGF receptor inhibitors in animal tumor models (Checkley at al., 2003). More recently, dynamic contrast enhanced MRI has been used to study the vascular response in clinical trials of patients with liver metastases treated with VEGF receptor inhibitors (Morgan at al., 2003). The limitations of these approaches are that response is limited to changes in tumor blood flow and the high cost of MIR scans. Disclosed herein are methods that can be used to develop peptides that bind to cells undergoing programmed cell death and necrosis. Peptides are selected that bind to apoptotic cancer cells as well as endothelial cells.

### Example 15

### Responses to Other TKIs

Pharmacodynamics is the study of the spatial and temporal response of biological tissue to a drug. In the case of cancer response to TKIs, tumors are biopsied or resected after TKI administration and the tumor response to the drug is assessed by histology. For example, TKIs that inhibit the PDGF receptor tyrosine kinase include SU6668, SU11248, and ST1571 (GLEEVEC®; a TKI that inhibits, inter alia, PDGFR and c-kit).

The response of cancer cells within the intracranial glioblastoma tumor model, GL261, was also tested in mice. GL261 tumors were implanted into the brains of C57BL/6 mice. After tumor formation (seven days later), mice were treated with GLEEVEC®, 4 Gy or both GLEEVEC® and 4 Gy. Tumors were sectioned and assayed with TUNEL stain to identify apoptotic nuclei. Tumors treated with the TKI alone showed 5% of glioma nuclei stained positive with TUNEL stain as compared to 6% following 4 Gy of radiation. Tumors treated with TKI followed by irradiation showed 18% apoptotic nuclei. The pharmacodynamic response (such as apoptosis) in tumors treated with GLEEVEC® correlated with tumor growth delay.

### Example 16

### Binding of Recombinant Phage Peptides to Responding Tumors

To determine the feasibility of imaging phage peptides binding within tumors, a Xenogen imaging system (Xenogen Corp., Alameda, Calif., United States of America) and near infrared imaging of Cy7-labeled recombinant peptides selected from phage libraries was employed. In order to examine the binding of recombinant phage peptides to responding tumors, preliminary experiments were performed to test the background binding of negative control phage in tumor-bearing animals. The selected phage HVGGSSV (SEQ ID NO: 1) was labeled with Cy7 and injected by tail vein into a mouse bearing tumors in both hind limbs. The mouse had been treated with VEGF receptor inhibitor (SU11248; 40 mg/kg), and the tumor in one hind limb exposed to 3 Gy 24 hours prior to imaging. Binding of the labeled peptide was observed in the responding tumor but not an non-responding tumor. The time course of labeled negative control phage circulating throughout the animal over 6 hours was determined. At 1 hour post-injection via the tail vein, Cy7-labeled phage was distributed throughout the entire animal. At 6 hours after tail vein injection, clearance through the kidneys was observed.

After determining that the background binding of the negative control phage was very low and was being substantially cleared from the animal, a control tumor was implanted into the right hind limb, and the mouse was not treated with SU11248 or radiation. At 24 hours following tail vein injection, there was minimal phage binding within the negative control tumor but residual binding within tail vein and kidney. In animals treated with SU11248 and 3 Gy to the right hind limb tumor, the Cy7-labeled phage peptide bound within the responding tumor indicating a physiologic response to therapy within that tumor. This binding was confirmed by histological and TUNEL analysis, which demonstrated that phage peptide binding correlated with tumor histology.

### Example 17

### A VEGF Receptor TKI, SU11248, Reduces the Threshold Radiation Dose Required for Peptide Binding

Recombinant peptide and ligand binding to the α_{2b}β₃ integrin is dose dependent, with a threshold dose of 6 Gy and maximal binding at 10 Gy. This is the dose range for induction of apoptosis within tumor endothelium (Garcia-Barros & Kolesnick, 2003). As disclosed herein, the threshold dose for induction of apoptosis was reduced to 2 Gy when the VEGF receptor TKI, SU11248, was administered prior to irradiation. To determine whether recombinant peptides bind within tumors following this combined therapy, the recombinant peptide SLRGDGSSV (SEQ ID NO: 2) was employed. The peptide was radiolabeled with ¹³¹I and injected by tail vein into mice bearing hind limb LLC tumors treated with 2 Gy and intraperitoneal SU11248 as described in Schueneman et al., 2003. Tumors were resected and counts per minute (CPM) were measured by well counts.

Tumors treated with SU11248 and 2 Gy bound 91% of radiolabeled peptide as compared to 9% and 10% bound with tumors treated with either SU11248 alone or 2 Gy alone (p<0.05). In comparison, tumors treated with 10 Gy bind 89% of peptide and 8% binds within untreated control tumors. Tumors were approximately 8% of body weight, indicating that 8% binding was expected in untreated control tumors.

### Discussion of Example 17

Apoptosis within the endothelium occurs following either treatment with high dose irradiation alone (10 Gy) or in response to the combination of RTK inhibitor and 2 Gy (Fuks et al., 1995; Schueneman et al., 2003). Studies of peptide binding within tumor blood vessels following 10 Gy in clinical trials have demonstrated that lower doses of radiation are not sufficient to initiate receptor activation when radiation is given alone (Hallahan et al., 2001b). More recent studies have shown that inhibitors of RTKs lower the threshold for radiation-induced injury within tumor microvasculature (Geng et al., 2001; Schueneman et al., 2003). As disclosed herein, phage displayed peptide libraries can be employed to select peptides that bind to tumor blood vessels following treatment with VEGF receptor antagonist combined with 2 Gy irradiation.

### Example 18

### In Vivo Panning of Recombinant Phage Binding to Tumors Treated with Radiation and TKIs

To identify additional peptides that bind within tumors treated with radiation and TKIs, in vivo panning of recombinant phage is performed. Tumors are implanted into the hind limb of mice and treated with 3 Gy and SU11248. 2 phage libraries are employed: the 17 phage linear and cyclic peptide libraries described in Hallahan et al., 2003 (provided by E. Ruoslhati of the Burnham Institute, La Jolla, Calif., United States of America). The background binding within tumor blood vessels is 10⁻⁴ for in vivo phage display. Phage are amplified so that 100 copies of each individual phage are present in the initial pool. The diversity of the library is 10⁷, so 10⁹ PFU are injected on the first round of biopanning. Phage recovered from responding tumors are then amplified so that all subsequent rounds of phage administration are in the range of 10⁹ PFU.

Phage libraries are administered by intracardiac injection at 24 hours following therapy. The mice are perfused with 10 ml of PBS into the left ventricle that is thereafter recovered from the right atrium. PBS is perfused at a rate of 2 ml per minute. Mice are sacrificed and organs and tumors are removed to quantify plaque-forming units. Organs are weighed so that the number of phage can be normalized by weight of the organ. Tissues are disrupted by use of hand held homogenizer on ice. The homogenizer is cleaned with bleach and rinsed between homogenization of different organs. Homogenate is then microcentrifuged at 5000 rpm and supernatant is discarded. The pellets are resuspended in 1% BSA and Modified Eagle Medium (MEM) and washed 5 times.

The T7 phage are then amplified using E. coli BL21 bacteria. The titer of T7 phage output from each organ and tissue is first measured by counting plaques within bacterial culture in agar plates. To determine the total phage output per organ, the number of plaque forming units on each plate is divided by the volume of phage that are plated and the weight of each organ. Phage are then amplified at 37° C. for 2 hours in BL21 until the culture is lysed and clarified. Cultures are then centrifuged at 8000 RPM for 15 minutes and filtered through 0.2 μm filter tipped syringes. A 2-fold difference in PFU in a particular organ is a normal variation.

Phage that bind to the vasculature within responding tumors show enrichment in the tumor relative to other organs and enrichment in the responding tumor relative to the control phage without DNA insert. These “homing phage” show a background in control organs that is lower than control phage without DNA insert.

PCR is used to amplify the recombinant phage insert coding region directly from the plaques. 50 clones are sequenced following 6 rounds of selection. Sequences that appear multiple times after 6 rounds of biopanning are identified. The PCR primer pair includes a T7 “up” primer, a 20-mer with the sequence AGCGGACCAGATTATCGCTA (SEQ ID NO: 20; Novagen). The T7 “down” primer is a 20-mer with the sequence AACCCTCAAGACCCGTTTA (SEQ ID NO: 21). The primer pair solution is prepared at 0.2 μmol/μl in water. PCR beads are dissolved in 24 μl of primer pair solution. Each T7 plaque is suspended in 10 μl of 1× Tris-buffered saline (TBS). The PCR reaction mixture is mixed with 1 μl of phage suspension. The sequencing reaction is performed and analyzed in an ABI PRISM® 377 DNA sequencer (Applied Biosystems, Foster City, Calif., United States of America). The 5′ flanking region translates to DPN in all recombinant peptides.

### Example 19

### Isolation of Peptides 24 Hours After Treatment

Recombinant peptides that bind within tumor blood vessels at 24 hours following therapy are selected from a cyclic peptide library using techniques similar to those disclosed hereinabove. Use of the cyclic peptide library increases the diversity of peptides that bind to responding tumor microvasculature, and the 24-hour time point increases the diversity of peptides recovered. RTK inhibition will reduce the threshold dose of radiation needed to induce recombinant peptide binding.

Moreover, the data presented herein indicated that apoptosis occurs within tumor vascular endothelium at 24 hours following treatment with SU11248 and radiation. The phage peptides disclosed hereinabove were originally isolated from tumors at 6 hours following treatment. The present Example is designed to study peptides that bind within tumor microvasculature during the onset of apoptosis.

Disclosed herein are recombinant peptides that bind within responding tumor microvasculature, rendering it possible to detect tumor vascular injury by use of phage displayed peptide libraries. The advantage in using phage displayed libraries for the selection of peptides is that posttranslational changes in preexisting molecules, and the unveiling of sequestered proteins can bind peptides. Both linear and cyclic peptide T7 phage libraries are employed because of the wide diversity of these libraries. This approach increases the likelihood of developing peptides with greater sensitivity and specificity for tumor response to therapy. By using both libraries and the 24-hour time point, increased numbers of peptides that bind to tumor microvasculature following treatment with SU11248 and radiation are identified.

### Example 20

### Prioritization of Recovered Phage

Selected phage could be bound nonspecifically to tumor proteins. These peptides are prioritized by sensitivity and specificity of binding to responding tumors. These peptides are validated and prioritized based on their tumor specific binding. Tumor blood flow is reduced at 5 days following combined treatment with TKIs and radiation (Donnelly et al., 2001). However, reduced blood flow has not been observed at 24 hours, which is merely the time of onset of vascular injury. To be certain that phage do not accentuate the effectiveness of therapy, blood flow is measured following the administration of phage libraries.

### Example 21

### Side-by-side Comparison of Selected Peptide Binding Within Responding Tumors

To determine which of the phage-displayed peptides bind most efficiently in tumors treated with combined VEGF receptor antagonist and radiation, tumors are implanted and treated as described herein (see also Geng at al., 2001; Edwards et al., 2002; Tan & Hallahan, 2004; Schueneman at al., 2003). SU11248 is given systemically. The right hind limb tumor is treated with irradiation (2 Gy). Because each phagemid DNA encodes a specific recombinant peptide on capsid proteins, it is possible to inject each of the phage that encodes peptides. Phage injection and tumor harvesting are performed as described in Hallahan at al., 2003. Tumors are resected from animals and each is weighed prior to homogenization. Phage are recovered separately from tumor and normal tissues and infected into bacterial cultures. The number of each phage recovered from responding tumor are counted and compared to the number of the same phage binding within the whole animal.

The phage peptides that achieve tumor specific binding are compared to previously characterized peptides (see Table 2). Phage are compared by simultaneous injection into the tail vein of the same mouse. Tumors are resected and phage peptides binding within responding tumor are compared to phage peptides binding within non-responding tumors and normal tissues. The DNA from recovered phagemid is sequenced as described in Example 18. The ratio of phage bound in responding tumors is compared to that recovered from non-responding tumors and normal tissues as described hereinabove.

As a control, an unirradiated (internal) control tumor is implanted into the left hind limb, and the right hind limb tumor is irradiated with 2 Gy following SU11248 administration. A second negative control includes a separate group of mice with two hind limb tumors, but receiving no SU11248. Again, the right tumor in each is irradiated and the left is an untreated internal control. These controls indicate whether peptides can be used to detect response to SU11248 alone or 2 Gy alone as compared to those that bind within only tumors treated with both agents. The negative control phage is a phage with a random peptide on its surface to determine whether phage are non-specifically trapped within tumors.

Phage colonies from tissue homogenates are amplified and sequenced as is described in Example 18. The ratio of phage peptides binding within tumors treated with SU11248 and radiation is compared to that in non-responding tumors, normal tissues, and tumors treated with single agents.

### Example 22

### Correlation of Peptide Binding with Apoptosis Within Tumor Vascular Endothelium Following Treatment with Radiation and VEGF Receptor TKIs

To determine whether the identified peptides detect tumor response to therapy, tumor tissue is studied by use of the identified peptides that bind to tumor blood vessels treated with VEGF receptor TKIs and radiation. Tumors are treated as described herein (see also Geng at aL, 2001; Schueneman et al., 2003; Lu et al., 2004) in each of the groups indicated below in Table 3. Control groups of mice treated with sub-therapeutic levels of TKI and/or radiation are employed in order to determine the specificity of peptide binding to only responsive tumors. Peptides are tagged with a FLAG epitope tag and a biotin tag. Each of the tags are studied separately in order to minimize artifacts such as nonspecific binding of peptides within unresponding tumors and normal tissues. Once it has been determined which tagging method produces minimal nonspecific binding, this tag is employed to study additional peptides. At 24 hours following treatment, peptides are administered by tail vein injection as described in (Hallahan et al., 2003). When peptides are cleared from the circulation is determined. It is expected that peptides clear within 2 hours, at which time mice are sacrificed and tumors are sectioned in half for both formalin fixation and freezing.

TABLE 3
Treatment Groups

| Group Number | Peptide |
|---|---|
| 1. untreated control | Identified peptide |
| 2. SU11248 alone | Identified peptide |
| 3. Radiation alone | Identified peptide |
| 4. Sub-therapeutic SU11248 + rad | Identified peptide |
| 5. Sub-therapeutic rad + SU11248 | Identified peptide |
| 6. SU11248 + radiation | Identified peptide |
| 7. SU11248 + radiation | random sequence peptide |
| 8. SU11248 + radiation | no peptide |

Tumor sections are co-stained with antibody to the FLAG epitope tag present on peptides and TUNEL staining for apoptosis in tumor sections as has been described herein (see also Schueneman et al., 2003; Hallahan et al., 2003). Both fluorescent probes and immunohistochemistry (IHC) probes are employed to study co-localization of peptides with tumor endothelium and with apoptotic cells using microscopy. Endothelium is stained with antibodies to CD31 and/or von Willebrand Factor (vWF). Apoptosis is detected by TUNEL, which has been effective at detecting endothelial apoptosis following treatment with SU11248 and radiation (Schueneman et al., 2003).

If nonspecific binding or absence of binding is observed, peptides are conjugated directly to fluorescent particles such as Quantum Dots (Quantum Dot Corp., Hayward, Calif., United States of America), or to Cy3/5. One goal is to study a peptide that is likely to be used in clinical imaging studies, so tagged peptides that can be detected by IHC are initially employed. Radiolabeled peptide binding to tumor regression is correlated. The random sequence peptide is tagged with the same tag so that it can be determined if the tag influences peptide binding patterns. Although unlikely, the peptides could accentuate the biological response to therapy. Therefore, a control group receiving SU11248, radiation, and no peptide is included.

To reduce the probability that peptide detection of response is tumor-type or mouse-strain specific, peptides are assessed in three tumor models in two strains of mice: B16F0 and LLC tumors in C57BL/6, mice and H460 tumors in nude mice. These additional tumor models are studied using peptides identified using the techniques disclosed herein.

### Example 23

### Correlating Peptide Binding with Tumor Growth Delay

To determine whether peptides detect tumor susceptibility to treatment with TKIs and radiation, peptide binding to responding tumors is correlated to tumor regression. Tumors are implanted into the hind limb and treated as described hereinabove. SU11248 is studied initially, but other VEGF receptor TKIs are also studied. SU11248 is administered by intraperitoneal injection and tumors are irradiated one hour later with 2 Gy. Radiolabeled peptides are injected by tail vein. The injected animals are imaged at varying time intervals. The pattern and level of peptide binding are analyzed as described herein. Mice are thereafter treated daily with SU11248 and radiation as described herein (see also Schueneman et al., 2003; Geng et al., 2001; Lu et al., 2004). To reduce the probability that peptide detection of response is tumor-type or mouse-strain specific, peptides are studied in three tumor models in two strains of mice: B16F0 and LLC tumors in C57BL/6 mice, and H460 tumors in nude mice.

### Example 24

### Specific Binding of Radiolabeled Peptides within Responding Tumors

The T7 phage has 415 copies of the same peptide on its surface. This polyvalence of the T7 phage could result in improved binding in peptides displayed on the T7 phage. Peptides are produced synthesized and the correct amino acid sequences of the peptides are verified. Each peptide has a unique amino acid sequence and unique molecular weight that can be used as a tool to determine which peptide has the greatest binding as measured on the mass spectrometer. The mass spectrometer is only semi-quantitative. A more quantitative approach is to immunoprecipitate peptides by use of the antibody to a FLAG tag on peptides. Tumors and whole animal homogenate are immunoprecipitated by the anti-FLAG tag antibody. The precipitated peptides are then sequenced using previously described sequencing techniques in tandem mass spectrometry (Liebler et al., 2002).

The phage peptides that bind most specifically to responding tumors are determined. Peptides are synthesized and radiolabeled with ¹⁸F or ¹³¹I. The binding of radiolabeled peptides is quantified by use of both non-invasive imaging and well counts. Peptides containing tyrosine residues not associated with the active binding site of the peptide can be labeled directly with radioiodine by electrophilic radioiodination in the presence of Chloramine-T (N-chloro-p-toluene sulfonamide sodium salt) or IODO-GEN® (Greenwood et al., 1963; Farah & Farouk, 1998). Histidine can also be iodinated directly, with some modifications of conditions, albeit not as efficiently (Gotthardt, 2002). For more general application, the radiolabel is introduced by conjugation of the peptide to a prosthetic group, which can itself be radiolabeled. Each requires control experiments to verify that the conjugate retains binding and pharmacokinetic properties. Variations in the prosthetic group itself, variable linker or tether molecular segments, and choice of site of conjugation on the peptide allow tailoring the properties of the radiotracer (Wust et al., 2003). For instance, ¹⁸F can be introduced via a fluorobenzoate conjugate; fluorobenzoic acid is first prepared by nucleophilic exchange with an activated precursor (trimethylammonium- or nitrobenzoic acid) and then coupled to the amino acid's amino group (or to an exposed lysine residue; Okarvi, 2001). ¹²³I or ¹³¹I can be introduced in the same fashion via iodobenzoic acid or 3-iodo-4-hydroxybenzoic acid; for this application, it is possible to iodinate an active ester, N-hydroxysuccinimidyl-4-hydroxybenzoate (Bolton-Hunter reagent) directly, followed by coupling with the peptide (Greenwood et al., 1963; Russell et al., 2002). Radioactive metals, such as ^{99m}Tc and ¹¹¹In, are attached by complexation with a chelating moiety conjugated to the target peptide.

That the peptide maintains affinity for surface peptides is verified by BIACORE® assessment of affinity of peptides for target protein. Peptide binding is also assessed within mice bearing tumors treated with TKIs and radiation. The whole animal is imaged as described in Hallahan et al., 2003). In addition, tumors are dissected from the animal and the amount of radiolabeled peptide in tumor and whole body are measured. Peptide binding within tissues is verified using immunohistochemistry to the FLAG tag on peptides.

### Example 25

### Recombinant Peptide Binding Within Tumors Treated with Other VEGFR Antagonists

To determine whether recombinant peptide binding within tumors is generalized to other VEGF receptor antagonists, tumors are implanted and treated as described herein (see also Geng et al., 2001; Edwards et al., 2002; Tan & Hallahan, 2004; Schueneman et al., 2003). VEGF receptor inhibitors that are in clinical trials and that are individually tested include AEE788, PTK787, ZD6474, and SU6668, each of which is given systemically to tumor-bearing mice. These agents are prioritized based on safety and efficacy in clinical trials. Other VEGFR inhibitors are studied as they progress in clinical trials.

The right hind limb tumor is treated with irradiation. Because each phagemid DNA encodes a specific recombinant peptide on capsid proteins, each of the phage that encode peptides identified as described herein can be injected. Phage injection and recovery is performed as described in Example 2. Briefly, tumors are resected from animals and each is weighed prior to homogenization. Phage are recovered separately from tumor and normal tissues and infected into bacterial cultures. The number of each phage recovered from responding tumor is compared to the number of the same phage binding within the whole animal are counted.

### Discussion of Examples 24-25

The treatment structure for Examples 24-25 can be found in Table 4. In Example 24, each mouse is implanted with two tumors, and randomly assigned either SU11248 or control. Further, one tumor from each mouse is treated with irradiation. Thus, TKI application is a “whole-mouse” level factor, while irradiation is a tumor within a mouse factor. This mixture of experimental units creates a slight complication to analysis since the effect of irradiation is estimated “within mouse” while the effect of the TKI SU11248 is inter-mouse. In Example 25, the same procedure is followed as in Example 24, except that the TKIs AEE788, PKT787, ZD6474, or SU6668 are employed instead of SU11248.

TABLE 4
Treatment Groups

| | Treatment | |
|---|---|---|
| | Labels | Treatment Groups |
| Example 24 | 24.1 | Control - tumor with no treatment using |
| | | radiolabeled peptides |
| | 24.2 | Tumor with radiation alone using |
| | | radiolabeled peptides |
| | 24.3 | Tumor with SU11248 alone using |
| | | radiolabeled peptides |
| | 24.4 | Tumor with SU11248 and radiation using |
| | | radiolabeled peptides |
| | 24.5 | Whole body (treated systemically with |
| | | SU11248) using radiolabeled peptides |
| Example 25 | 25.1 | Tumor treated with radiation and AEE788 |
| | 25.2 | Tumor treated with radiation and PKT787 |
| | 25.3 | Tumor treated with radiation and ZD6474 |
| | 25.4 | Tumor treated with radiation and SU6668 |

Labeling the amino terminus of peptides should not interfere with peptide binding to inducible surface proteins in tumor vascular endothelium. Upon confirmation of this, the peptide is further developed using techniques described herein. If, however, radiolabeling peptides is found to reduce affinity for inducible molecules, nanoparticles are used for peptide conjugation. This approach is analogous to displaying the peptides on the surface of phage. For that matter, radiolabeled phage can be employed to test the hypothesis that polyvalent peptides on a core surface improve specific binding to responding tumors.

Peptide detection of tumor vascular responsiveness to TKIs can be generalized to all VEGFR inhibitors, or can be focused on specific examples, such as SU11248. Considering that peptides are binding to molecules that participate in physiologic response to vascular injury, it is most probable that peptides are useful in detecting response to all VEGF receptor TKIs.

### Example 26

### PET Imaging of ¹⁸F-labeled Peptides

In order to arrive at an appropriate imaging protocol using PET imaging of ¹⁸F labeled peptides, the relationship between the fractional-uptake of ¹³¹I labeled peptides in tumor bearing mice treated with TKI and radiation is determined. This relationship, including the determination of the time-point for optimal imaging, is determined using serial pinhole scintillation camera images. Confirmation studies at the previously determined optimal time-point using SU11248 and radiation are then performed using a microPET system (FOCUS, Concorde MicroSystems, Knoxville, Tenn., United States of America). The microPET results are used to define the initial protocols. ¹³¹I is employed as the radiolabel in these mouse studies because of the relatively long physical half-life needed for the kinetic studies (half-life of ¹³¹I is 8 days). ¹³¹I Imaging and Kinetics Measurements. Each mouse has identical implanted tumors in each flank. When tumors have achieved diameter of at least 5 mm, the left hind limb tumor is identified as the control side and does not receive radiation therapy. The right tumor is treated with 3 Gy. 50 μCi of ¹³¹I labeled peptide is injected via tail vein followed by serial pinhole images with the scintillation camera. Injection is made with the animal under the camera followed by dynamic image acquisition (12 images×5 minutes/image) for the first hour. Each animal is re-imaged at 2, 4, 8, 12, and 24 hours. The initial image (summed over the first 60 minutes) serves as the 100% dose reference image. Corrected for both acquisition time and radiation decay, all subsequent images are analyzed to provide percent of injected dose in both control and responding tumors, as well as “rest-of-the-body”. Following the 24-hour image, each animal is sacrificed and submitted to well-counter activity assessment of each tumor, as well as dissected organs (lung, kidneys, spleen and liver) for confirmation of relative distribution.

Radiolabeling peptides with ¹⁸F or ¹³¹I could reduce the affinity of peptides for target molecules. If reduced binding is observed, a number of different strategies are employed to determine whether this reduced affinity can be resolved. First, the radiolabel is linked to a linker at the terminus of peptide. A second strategy is to link peptides to a radiolabeled nanoparticle. The simplest nanoparticle would be to use the phage displayed peptides. Therefore, the phage is labeled prior to administration.

The ¹⁸F labeled peptide tumor affinity and kinetics might not be found to be identical to the ¹³¹I agent. In this circumstance, a complete dose response relationship at all radiation dose levels is repeated in a manner identical to the previously described ¹³¹I studies.

The valence of single peptides is 1, as compared to 415 copies of the same peptide on the T7 phage. Therefore, the phage are essentially polyvalent nanoparticles with peptides on the surface. As such, greater tumor specific binding might be achieved by phage whereas single peptides might show less specific binding. An alternative approach is to radiolabel phage that display to peptides on their surface. This allows for testing the alternative hypothesis that polyvalent peptide-coated particles improve sensitivity for imaging tumor response.

The peptides can be digested by peptidases in serum and tissue. Whether or not peptidases cause peptide degradation is determined by radiolabeling, and peptide fragments can be detected by mass spectrometry (Vanderbilt Proteomics Shared Resource, Vanderbilt University, Nashville, Tenn., United States of America). This can be addressed by conjugation of peptides to macromolecules such as nanoparticles as previously described in Hallahan et al., 2003.

### Example 27

### Differentiating Responding from Non-responding Tumors

To differentiate responding cancers from non-responding cancers following treatment with TKIs, a tumor that does not respond to the TKI SU11248 was studied, and peptide binding within this tumor was compared to that of a responding tumor (LLC). D54 and LLC tumors were implanted into both hind limbs of nude mice as described in Example 2. Tumors were grown over the course of seven to 10 days. Animals were then treated with SU11248, with or without 3 Gy irradiation.

At 24 hours following drug administration, mice were injected with Alexfluor 750-conjugated HVGGSSV (SEQ ID NO: 1) peptide through a jugular catheter. Labeled peptide binding was compared within untreated tumors in a first mouse to that of a second mouse that was treated with SU11248 and radiation to the left hind limb tumor. Intense peptide binding within the tumor treated with the combination of SU11248 and radiation was observed.

Two additional mice, mouse 3 and mouse 4, were treated with SU11248 alone. Tumors did not respond to therapy and show no increase in peptide binding following treatment with SU11248 alone. In comparison, LLC tumors responded to SU11248 alone or in combination with radiation. LLC tumors showed a tumor growth delay when treated with drug alone, whereas D54 tumors did not show tumor growth delay when treated with SU11248 alone.

Determinations of uptake in tumors treated with SU11248 alone as compared to untreated tumors indicated that peptide differentially bound to tumors that responded to SU11248 therapy compared to tumors that did not respond to therapy.

To study the kinetics of peptide binding to treated tumors, animals were imaged daily following administration of Cy 7 conjugated HVGGSSV (SEQ ID NO: 1) peptide. Peptide was observed to circulate throughout the entire mouse model over the course of 28 hours. At 40 hours, the peptide was excreted by the kidneys. Tumors were located in the left hind limb. Peptide began to bind to the tumor within 40 to 47 hours following SU11248 therapy. The peptide remained bound to the tumor over the course 162 hours.

To determine whether peptide binds to endothelium or blood components, tumors were sectioned at 24 hours following administration of biotinylated HVGGSSV (SEQ ID NO: 1) peptide. Tumor sections were then stained with strepavidin conjugates for histochemistry. Peptide was observed bound primarily to tumor vascular endothelium with minimal or no binding within the intravascular blood components.

### Example 28

### Identification of Receptors in Lung Cancer Cells that Bind to SEQ ID NO: 1

A Phage display library that displays the human cDNA from lung cancer cells was expressed on the g3p protein of T7 phage. This phage displayed protein library was incubated with the HVGGSSV (SEQ ID NO: 1) peptide. Putative receptors that bind to the HVGGSSV (SEQ ID NO: 1) peptide were selected. Potential receptors that bind to this ligand are identified by RT-PCR.

### REFERENCES

The references listed below, as well as all references cited in the specification, are incorporated herein by reference in their entireties to the extent that they supplement, explain, provide a background for, or teach methodology, techniques, and/or compositions employed herein.
Alexay et al. (1996) The PCT International Society of Optical Engineering 2705/63.
Amemiya et al. (1988) A Storage Phosphor Detector (Imaging Plate) and its Application to Diffraction Studies Using Synchroton Radiation. Topics in Current Chemistry, vol. 147, Springer-Verlag, Heidelberg, Germany.
Andersson et al. (2000) Biopolymers 55:227-250.
Bauminger & Wilchek (1980) Methods Enzymol 70:151-159.
Allen et al. (1995) Trends Biochem Sci 20:511-516.
Bendixen et al. (1994) Nucleic Acids Res 22:1778-1779.
Bodanszky (1993) Principles of Peptide Synthesis. 2nd rev. ed. Springer-Verlag, New York, United States of America.
Brenner & Lerner (1992) Proc Natl Acad Sci USA 89:5381-5383.
Brent & Finley (1997) Annu Rev Genet. 31:663-704.
Carninci et al. (1996) Genomics 37:327-33.
Chattopadhyay et al. (2001) Nucl Med Biol 28:741-744.
Checkley et al. (2003) Magn Reson Imaging 21:475-82.
Cheng (1996) Hum Gene Ther 7:275-282.
Cheng et al. (1994) J Med Chem 37:1-8.
Coatney (2001) Ilar J 42:233-247.
Cohen et al. (1998) Proc Natl Acad Sci USA 95:14272-14277.
Corringer et al. (1993) J Med Chem 36:166-172.
Deutscher (1990) Guide to Protein Purification. Academic Press, San Diego, United States of America.
Dewanjee et al. (1994) J Nucl Med 35:1054-1063.
Donnelly et al. (2001) Radiology 219:166-70.
Edwards et al. (2002) Cancer Res 62:4671-7.
European Patent No. 0 439 095
Farah & Farouk (1998) J Labelled Compd Radiopharm 41:255-259.
Fields & Noble (1990) Intl J Pept Protein Res 35:161-214.
Fields & Song (1989) Nature 340:245-246.
Forrer et al. (1999) Curr Opin Struct Biol 9:514.
Fraser (1996) Methods Cell Biol 51:147-160.
Fuks et al. (1995) Cancer J Sci Am 1:62.
Fuller et al. (1998) Biotechniques 25:85-88, 90-82.
Garbay-Jaureguiberry et al. (1992) Intl J Pept Protein Res 39:523-527.
Garcia-Barros & Kolesnick (2003) Science 300:1155-9.
Geng et al., (2001) Cancer Res 61:2413-2419.
George (2001). Semin Oncol 28(5 Suppl 17):27-33.
Goldman et al. (1997) Cancer Res 57:1447-1451.
Gorski et al. (1999) Cancer Res 59:3374-8.
Gotthardt (2002) Eur J Nucl Med 29:597-606.
Greenwood et al., (1963) Biochem J 89:114-23.
Hallahan & Virudachalam (1999) Radiat Res 152:6-13.
Hallahan et al., (1995a) Biochem Biophys Res Commun 217:784-795.
Hallahan et al. (1995b) Nat Med 1:786-791.
Hallahan et al. (1996) Cancer Res 56:5150-5155.
Hallahan et al. (1998) Cancer Res 58:5216-5220.
Hallahan et al. (2001a) J Control Release 74:183-191.
Hallahan et al. (2001b) Am J Clin Oncol 24:473-80.
Hallahan et al. (2003) Cancer Cell 3:63-74.
Hawiger & Timmons (1992) Meth Enzymol 215:228-243.
Hawiger et al. (1989) Biochemistry 28:2909-2914.
Healy et al. (1995) Biochemistry 34:3948-3955.
Heredia et al. (1991) J Neurosci Methods 36:17-25.
Hnatowich et al. (1996) J Pharmacol Exp Ther 276:326-334.
Julien et al. (2000) Biochemistry 39:75-85.
Kolonin & Finley (1998) Proc Natl Acad Sci USA 95:14266-14271.
Koivunen et al. (1993) J Biol Chem 268:20205-202.
Koivunen et al. (1994) J Cell Biol 124:373-38.
Lecrenier et al. (1998) Bioessays 20:1-5.
Lees (2001) Semin Ultrasound CT MR 22:85-105.
Leibel & Phillips (1998) Textbook of Radiation Oncology, Saunders, Philadelphia, United States of America.
Liebler et al. (2002) Anal Chem 74:203-210.
Licha et al., (2000) Photochem Photobiol 72:392-398.
Lu at al. (1995) Biotechnology (NY) 13:366-372.
Lu et al. (2004), Intl J Radiat Oncol Biol Phys 58:844-50.
McOmie (1973) Protective Groups in Organic Chemistry. Plenum Press, New York, United States of America.
Mendel et al., (2002) Clin Cancer Res 9:327-37.
Merrifield (1969) Adv Enzymol Relat Areas Mol Biol 32:221-296.
Morgan at al. (2003) J Clin Oncol 21:3955-64.
Nabel (1997), Current Protocols in Human Genetics. John Wiley & Sons, New York, United States of America.
Neri et al. (1997) Nat Biotechnol 15:1271-1275.
Okarvi (2001) Eur J Nucl Med 28:929-38.
Paris at al. (2001) Science 293:293-297.
Park at al. (1997) Adv Pharmacol 40:399-435.
Pasqualini et al. (1997) Nat Biotechnol 15:542-546.
Pavone at al. (1993) Intl J Pept Protein Res 41:15-20.
PCT International Publication No. WO 01/09611.
Pierschbacher & Ruoslahti (1987) J Biol Chem 262:17294-17298.
Pomper & Port (2000) Magn Reson Imaging Clin N Am 8:691-713.
Prewett et al. (1999) Cancer Res 59:5209-18.
Ragnarson et al. (1992) Histochemistry 97:329-333.
Rehrauer et al. (1996) J Biol Chem 271:23865-23873.
Rovaris at al. (2001) J Neurol Sci 186 Suppl 1:S3-9.
Ruoslahti (1996) Annu Rev Cell Dev Biol 12:697-715.
Russell et al. (2002) J Nucl Med 43:671-7.
Sagiuchi et al. (2001) Ann Nucl Med 15:267-270.
Sambrook & Russell (2001), Molecular Cloning: A Laboratory Manual, Third Edition, Cold Spring Harbor Laboratory Press, Cold Spring Harbor, N.Y., United States of America.
Schneider & Eberle (1993) Peptides, 1992: Proceedings of the Twenty-Second European Peptide Symposium, Sep. 13-19, 1992, Interlaken, Switzerland, Escom, Leiden.
Schröder & Lübke (1965) The Peptides. Academic Press, New York, United States of America.
Schueneman et al., (2003) Cancer Res 63:4009-4016.
Schwendener (1992) Chimia 46:69-77.
Seghezzi et al. (1998) J Cell Biol 141:1659-73.
Shen et al. (1993) Magn Reson Med 29:599-604.
Smith (1985) Science 228:1315-1317.
Smith (1997) Clinical Rev 97:391.
Staba et al. (2000) Cancer Gene Ther 7:13-19.
Stewart & Young (1969) Solid Phase Peptide Synthesis. Freeman, San Francisco, United States of America.
Tan & Hallahan (2004) Cancer Res 63:7663-7.
Tang & Casey (1999) Biochemistry 38:14565-14572.
Tavitian et al. (1998) Nat Med 4:467-471.
Tsai et al. (1995) J Neurosurg 82:864-73.
Tung et al. (1992) Pept Res 5:115-118.
U.S. Pat. Nos. 4,244,946; 5,088,499; 5,147,631; 5,168,037; 5,223,409; 5,264,563; 5,498,538; 5,578,629; 5,650,489; 5,667,988; 5,702,892; 5,707,605; 5,738,837; 5,738,996; 5,747,334; 5,756,291; 5,780,225; 5,811,392; 5,811,512; 5,811,515; 5,817,757; 5,817,879; 5,824,483; 5,840,479; 5,858,670; 5,865,754; 5,922,545; 5,928,627; 5,948,635; 6,015,561; 6,015,881; 6,024,938; 6,031,071; 6,057,098; 6,071,890; 6,068,829; 6,080,384; 6,083,486; 6,107,059; 6,156,511; 6,159,443; 6,168,912; 6,174,708; 6,180,348; 6,214,553; 6,221,018; 6,231,834; 6,245,318; 6,246,901; and 6,254,852.
Urge et al. (1992) Carbohydr Res 235:83-93.
Vasavada et al. (1991) Proc Natl Acad Sci USA 88:10686-10690.
Vinogradov et al. (1996) Biophys J 70:1609-1617.
Weissleder et al. (1992) Magn Reson Q 8:55-63.
Weissleder et al. (1999) Nat Biotechnol 17:375-378.
Witte et al. (1989) Cancer Res 49:5066-72.
Wust et al. (2003) Appl Radiat Isot 59:43-8.
Yang et al. (1995) Nucleic Acids Res 23:1152-1156.
Yoo et al. (1997) J Nucl Med 38:294-300.
Zwick et al. (1998) (1998) Curr Opin Biotechol 9:427-436.

It will be understood that various details of the presently described subject matter can be changed without departing from the scope of the presently described subject matter. Furthermore, the foregoing description is for the purpose of illustration only, and not for the purpose of limitation.

1-98. (canceled)

99. A method of assessing the effectiveness of a treatment on a target, the method comprising:
(a) treating a tumor with at least one of ionizing radiation and an agent selected from the group consisting of a receptor inhibitor and a receptor tyrosine kinase inhibitor (TKI);
(b) contacting the target with a peptide that binds to the target, wherein the peptide comprises any of SEQ ID NOs: 1-18; and
(c) determining an extent of binding of the peptide to the target; wherein the extent of binding to the target correlates with the effectiveness of the treatment.

100. A method of noninvasive imaging of a cell undergoing apoptosis, the method comprising:
(a) binding to a cell undergoing apoptosis a reagent that binds to a molecule induced by apoptosis, the reagent comprising:
(i) a peptide the binds to a tumor treated with at least one of ionizing radiation and an agent selected from the group consisting of a receptor inhibitor and a receptor tyrosine kinase inhibitor (TKI), wherein the peptide comprises any of SEQ ID NOs: 1-18; and
(ii) a contrast agent; and
(b) detecting the binding of the reagent to the cell, whereby a cell undergoing apoptosis is imaged.

101. The method of claim 99, wherein the target is selected from the group consisting of a molecule induced on a tumor cell, an endothelial cell associated with tumor vasculature, a blood component, and an apoptotic cell.

102. The method of claim 99, wherein the target is a receptor activated during a physiologic response in the subject to radiation treatment, receptor inhibitor treatment, TKI treatment, or a combination thereof.

103. The method of claim 99, wherein the peptide comprises at least one of SEQ ID NOs: 1-8.

104. The method of claim 99, wherein the tumor is a glioma, a melanoma, a lung tumor, or a metastasis therefrom.

105. The method of claim 99, wherein the agent is selected from the group consisting of a vascular endothelial growth factor (VEGF) antagonist, a Her-2/ErbB2 antagonist, an epidermal growth factor receptor (EGFR) antagonist, a platelet-derived growth factor receptor (PDGFR) antagonist, or a combination thereof.

106. The method of claim 105, wherein the agent comprises a monoclonal antibody.

107. The method of claim 105, wherein the determining step further comprises comparing an extent of binding of the peptide before the agent contacts the target with an extent of binding of the peptide after the agent contacts the target.

108. The method of claim 100, wherein the cell is present in a subject and the binding step comprises administering the peptide to the subject in an amount and by a route sufficient to deliver the peptide to the cell.

109. The method of claim 100, further comprising treating the tumor with at least one of ionizing radiation, a receptor inhibitor, and a TKI under conditions sufficient to induce apoptosis in the cell.

110. The method of claim 109, wherein the treating step is performed before the binding step.

111. The method of claim 110, wherein the treating is with a receptor inhibitor, a TKI, or both.

112. The method of claim 110, where the treating is performed about 24 hours prior to the binding step.

113. The method of claim 100, wherein the cell undergoing apoptosis is a tumor cell or a cell of a tumor-associated vascular network.

114. The method of claim 100, wherein the peptide comprises at least one of SEQ ID NOs: 1-8.

115. The method of claim 100, wherein the tumor is a glioma, a melanoma, a lung tumor, or a metastasis therefrom.

116. The method of claim 100, wherein the agent is selected from the group consisting of a vascular endothelial growth factor (VEGF) antagonist, a Her-2/ErbB2 antagonist, an epidermal growth factor receptor (EGFR) antagonist, a platelet-derived growth factor receptor (PDGFR) antagonist, or a combination thereof.

117. The method of claim 100, wherein the molecule induced by apoptosis is present on or in a tumor cell, an endothelial cell associated with tumor vasculature, a blood component, or combinations thereof.

118. The method of claim 100, wherein the peptide comprises at least one of SEQ ID NOs: 1-8.

119. The method of claim 100, wherein the tumor is a glioma, a melanoma, a lung tumor, or a metastasis therefrom.

120. The method of claim 100, wherein the detecting comprises a technique selected from the group consisting of Single Photon Emission Computed Tomography (SPECT), Positron Emission Tomography (PET), Magnetic Resonance Imaging (MRI), Fluorescent Imaging, and Near-infrared (NIR) Emission Spectroscopy.

121. The method of claim 120, wherein the peptide comprises a radiolabel, a fluorescent label, or both.
